(19) United States
(12) Patent Application Publication
(10) Pub. No.: US 2009/0280565 A1
(43) Pub. Date: Nov. 12, 2009

(54) HIGH-RATE PERFUSION BIOREACTOR

(75) Inventors: Mario Jolicoeur, St. Bruno (CA); Robert Legros, Kirkland (CA); Caroline De Dobbeleer, Rixensart (BE); Steve Hisiger, Montreal (CA)

Correspondence Address: CHOATE, HALL & STEWART LLP, TWO INTERNATIONAL PLACE, BOSTON, MA, 02110, US

(73) Assignee: CORPORATION De L'ECOLE POLYTECHIQUE MONTR'EAL, Montreal (CA)

(21) Appl. No.: 12/158,676

(22) PCT Filed: Dec. 22, 2006
(86) PCT No.: PCT/CA06/02131

§ 371 Date: Jan. 5, 2009

Related U.S. Application Data
(60) Provisional application No. 60/752,377, filed on Dec. 22, 2005.

(51) Int. Cl. C12N 5/04; C12N 5/02; C12M 1/00; C12M 1/12

(52) U.S. Cl. 435/412; 435/414; 435/420; 435/424; 435/425; 435/289.1; 435/297.1; 435/303.1

(57) ABSTRACT

The present invention relates to a novel perfusion bioreactor allowing continuous medium feed and extraction of metabolites or other desired products from cells. The invention is useful for plant cell cultures but may also be used for mammalian cell cultures, insect cell cultures and bacterial cell cultures. The design of the reactor includes sedimentation columns mounted inside the bioreactor to separate single cells and cell aggregates from the culture medium at a very low shear stress. The operating conditions allow a stable cell/medium separation by maintaining the medium upward velocity equal to or slightly lower than the cell sedimentation velocity.

### FIELD OF THE INVENTION

The present invention relates to a system and method for producing and/or isolating metabolites or other desired products from cells such as recombinant or native proteins. More specifically, the present invention relates to a high-rate perfusion bioreactor comprising an efficient cell/medium separation device allowing for continuous medium feed and extraction of metabolites or other desired products from cells.

### BACKGROUND OF THE INVENTION

Rapid and efficient removal of products generated by cells is particularly desirable when the product is associated with toxicity towards the cells and/or is itself unstable.

Mammalian cells and bacterial cells are often chosen for the production of recombinant proteins. However, production of recombinant proteins in insect cells or plant cells represents also a new and interesting approach. For example, In vitro plant cell cultures are believed to have a high potential for the production of secondary metabolites that are of pharmaceutical interest (Ramachandra and Ravishankar, 2002). However, secondary metabolites that are released in a plant cell culture medium can be re-used by the cells and be altered by diverse enzymes present in the cells' environment. Consequently, many approaches have been developed to remove metabolites of interest in situ. In the case of hairy roots, which can be easily confined into a bioreactor, a two-liquid phase bioreactor with the circulation of silicon oil was shown to be highly efficient for the continuous extraction of intracellular alkaloids (Tikhomiroff et al., 2002). For cell suspension systems, the use of an extraction phase was shown to protect secondary metabolites from degradation (Lee-Parsons and Shuler, 2002) in addition to resulting in an increased productivity (Williams et al., 1992, Lee-Parsons and Shuler, 2002). More recently, it has been shown that extractive XAD-7 resin can be placed within an external medium recirculation loop, without contacting the cells, which significantly improves metabolite harvesting and purification (Klvana et al., 2004). However, continuous cell separation from culture medium is not easy to achieve, and the numerous approaches developed for mammalian cells (Voisard et al., 2003), such as filtration (Kawahara et al., 1994), centrifugation (Johnson et al., 1996), sedimentation (Batt et al., 1990; Searles et al., 1994; Hülscher et al., 1992) and acoustic separation (Gorenflo et al., 2003) do not lend themselves to application to plant cells.

The chief constraints in the design of a cell/medium separator are: a high SCV reached in plant cell suspensions (close to 100%; Jolicoeur et al., 1992), cell shear sensitivity (Doran, 1999) and aggregate size distribution (Tanaka, 2000). The use of a nylon or a stainless-steel mesh was successful for the retention of cells, allowing continuous medium feed in flask cultures of Taxus cupidata (Seki et al., 1997) and Eschscholtzia californica (Klvana et al., 2004) at a maximum dilution rate of 1.0 d⁻¹ and a cell suspension of 4.9 d⁻¹, respectively. Spin or rotating filters have been studied to retain suspension plant cells in diverse bioreactor configurations. Hogue et al. (1990), Chattopadhyay et al. (2003) and Lee et al. (2004) reached maximum dilution rates of 0.25 d⁻¹, 1.8 d⁻¹ and 2.0 d⁻¹, respectively. However, as observed by Klvana et al. (2004), these systems are susceptible to filter clogging. Su et al. (1996, 2003) and Kim et al. (1991) have developed perfusion bioreactors that are based on cell sedimentation and do not required any membrane. Su et al. (1996) achieved a cell retention efficiency of 100% for a packed cell volume (PCV) of 20% but with no cell retention at a PCV of 60%, both at 1.0 d⁻¹ in an airlift bioreactor with a cell settling zone delimited by a baffle plate. Recently, Su and Arias (2003) reported a maximum perfusion rate reduced to 0.4 d⁻¹ to reach a cell retention efficiency of 100% at a PCV of 60% using an annular settling zone in a stirred-tank bioreactor. However, the goal of reaching high productivities may require both maximal cell concentration and perfusion rates that are higher than the production rate of the cells, otherwise negative feedback may be a significant factor, as suggested by Klvana et al. (2004).

Despite the above advances in cell bioreactor technology, there remains a need for a bioreactor that can produce metabolites more efficiently and in greater quantity. The present invention seeks to meet this and related needs.

### SUMMARY OF THE INVENTION

The present invention relates to a novel perfusion bioreactor allowing continuous medium feed and extraction of metabolites or other products from cells such as native or recombinant proteins. The present invention may be useful for mammalian cells, plant cells, insect cells and bacterial cells.

The present invention provides a perfusion bioreactor apparatus which may comprise, for example;
a vessel for receiving a cellular biomass suspension comprising cells and a cellular medium therein,
an extractor assembly mounted to the vessel for extracting the cellular medium from the vessel;
   wherein the extractor is adapted to extract the cellular medium substantially avoiding extraction of the agitated cellular biomass.

The bioreactor apparatus may also comprise an agitator assembly for so agitating the cellular biomass suspension within the vessel as to provide a stable sedimentation front.

More particularly, the present invention provides a perfusion bioreactor apparatus which may comprise:
a vessel for receiving a cellular biomass suspension which may comprise cells and a cellular medium therein,
an agitator assembly for so agitating the cellular biomass suspension within the vessel as to provide a stable sedimentation front;
an extractor assembly for extracting the cellular medium from the vessel;
   wherein when the cellular biomass suspension in the cellular medium is agitated within the vessel thereby, the extractor assembly may be adapted to extract the cellular medium while substantially avoiding extraction of the agitated cellular biomass.

In accordance with the present invention, when the cellular medium comprises cellular product, the extractor assembly may be adapted to extract the cellular medium along with the cellular product.

The perfusion bioreactor apparatus of the present invention may more specifically comprise:
a vessel which may be adapted to receive at least one sedimentation column and a cellular biomass suspension;
an agitator assembly;
air and/or gas entry means;
air and/or gas exit means;
liquid entry means; and
liquid exit means.

The apparatus may comprise a sedimentation column having a portion comprised within the vessel (inlet) and a portion comprised outside the vessel (outlet).

In accordance with an embodiment of the present invention, the perfusion bioreactor apparatus may be adapted for the continuous extraction of the cellular medium from the bioreactor. The apparatus may also be adapted for the continuous removal of cellular product from cell culture medium.

In accordance with an exemplary embodiment of the invention, the sedimentation column may be mounted in the vessel along the longitudinal length thereof.

In accordance with another embodiment of the present invention, the perfusion bioreactor apparatus may be adapted for a continuous feed of cell culture medium.

Also in accordance with an embodiment of the invention, the bioreactor apparatus and process may be used for growing cells and/or for producing a cellular product from cells, such as, for example, a metabolite, a native protein, a recombinant protein. It is to be understood herein that the cellular product is not intended to be limited to secreted proteins or metabolites. For example, cellular products may be released upon cell lysis, cell death, etc., and the apparatus and process of the present invention may advantageously be used to isolate a desired product released in the cell culture medium.

Exemplary embodiments of cells which may be advantageously used with the bioreactor apparatus include for example and without limitation, mammalian cells, plant cells, insect cells and bacterial cells.

In accordance with the present invention, the cells may be grown in suspension or alternatively may be adherent cells grown on micro-carriers.

In one embodiment, the design of the reactor includes four sedimentation columns mounted inside a 2.5-l bioreactor to separate single cells and cell aggregates from the culture medium at a very low shear stress. Eschscholtzia californica cells were used as a model system for the production of secondary metabolites. The liquid medium free of cells and debris is continuously recirculated in the bioreactor via an external loop containing extraction columns comprising fluidized resin, such as XAD-7, for the adsorption of benzophenanthridine alkaloids. The operating conditions allowing a stable cell/medium separation inside the sedimentation system were determined from hydrodynamic studies. It was shown that a medium upward velocity equal to the cell sedimentation velocity maintained stable cell/medium separation front (a stable sedimentation front). A maximum dilution rate of 20.4 d⁻¹ was reached from day 4 to day 6 and it was then regularly reduced down to 5 d⁻¹ for the last day. The perfusion bioreactor was shown to be efficient for cultures of 10 and 14 days, with a cell suspension reaching a sedimented cell volume of 50%.

In accordance with the present invention, there is provided a perfusion bioreactor incorporating a simple and effective cell/medium separation device coupled with an external polymeric resin column for continuous extraction of secondary metabolites. Hydrodynamic and mass transfer studies leading to the determination of stable cell/medium separation operating conditions were performed using E. californica suspension cells as a model system. This system was further validated with a Nicotiana tabacum cell culture with an external affinity resin column for continuous extraction of recombinant proteins.

The present invention also provides in a further aspect, a process for the continuous extraction of a cellular product contained in a cell culture medium.

In accordance with an exemplary embodiment of the invention, the process may comprise, for example:
providing a cellular biomass suspension (which may comprise cells and cell culture medium) in agitation;
providing for the production of a cellular product from the cellular biomass suspension;
providing a stable sedimentation front;
extracting the cell culture medium with the cellular product from the cellular biomass suspension at an extraction velocity rate equal to or lower than a sedimentation velocity of the cellular biomass suspension thereby substantially avoiding extraction of the cellular biomass.

In accordance with a further embodiment the process may further comprise a step of providing for the separation of the cellular medium and the cells before extracting.

The process of the present invention may provide for the continuous removal of the cellular product from the cell culture medium.

In accordance with an embodiment of the present invention, the sedimentation velocity of the cellular biomass suspension may be determined in a linear region of a slope made by measuring a cell bed height decrease rate as a function of time.

In accordance with a further embodiment of the present invention, the sedimented cell volume may be used for determining the sedimentation velocity of the cellular biomass suspension.

In accordance with the present invention, the sedimented cell volume may be, for example 80% or less, 75% or less or alternatively 70% or less.

In accordance with the present invention, the sedimented cell volume may be, at least 80% or at least 90%.

The present invention also relates to a cellular product produced by the process described herein.

The present invention also relates to a cellular product extracted by the process described herein.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

In the appended drawings;

FIG. 1(a) is a schematic representing an exemplary embodiment of cell/medium separation devices of funnel configuration,

FIG. 1(b) is a schematic representing an exemplary embodiment of cell/medium separation devices of funnel configuration comprising an exemplary embodiment of a vortex reducer,

FIG. 1(c) is a schematic representing an exemplary embodiment of cell/medium separation devices of conical configuration,

FIG. 1(d) is a schematic representing an exemplary embodiment of cell/medium separation devices of conical configuration comprising an exemplary embodiment of a vortex reducer,

FIG. 2(a) is an isometric view of an exemplary embodiment of a perfusion bioreactor system in accordance with the present invention,

FIG. 2(b) is a top view of an exemplary embodiment of a perfusion bioreactor system in accordance with the present invention,

FIG. 2(c) is an isometric view of the second sedimentation module,

FIG. 3 (A) is a graph of an exemplary embodiment of sedimentation velocity (Uₛ) as a function of cell suspension sedimented cell volume (SCV),

FIG. 3 (B) is a graph of an exemplary embodiment of cell sedimentation time profile of a 4-days old 100 ml suspensions from flasks,

FIG. 3 (C) is a graph of an exemplary embodiment of the variation of the sedimentation velocity cell as a function of cell suspension sedimented cell volume (SCV),

FIG. 4 is a graph illustrating in an exemplary embodiment, the influence of air flow rate on K_{L}a: (□) 60 rpm, clockwise (downward pumping), sparger installed at the bottom of the bioreactor; (◯) 60 rpm, counter clockwise (upward pumping), sparger installed 6 cm from the liquid surface (top); (⋄) 45 rpm, counter clockwise, top sparger; (▪) 60 rpm, clockwise, top sparger,

FIG. 5 (A) is a graph illustrating the cell growth index for the different bioreactor cultures. (□) bioreactor culture without extractive phase; (Δ) bioreactor culture with free resins; () perfusion bioreactor culture with antifoam; (▾) perfusion bioreactor culture without antifoam. Elicitation: time of elicitation with the chitin solution,

FIG. 5 (B) is a graph illustrating the dry weight with time for the different bioreactor cultures. (□) bioreactor culture without extractive phase; (Δ) bioreactor culture with free resins; () perfusion bioreactor culture with antifoam; (▾) perfusion bioreactor culture without antifoam. Elicitation: time of elicitation with the chitin solution,

FIGS. 6 (A), (B) and (C) are histograms illustrating the intracellular alkaloid production for the different bioreactor cultures. Dense right slanted dash=sanguinarine, sparse left slanted dash=chelerythrine, light gray=chelerubine, white=chelilutine, dark gray=macarpine. (A) Bioreactor without extractive phase; (B) bioreactor with suspended resin; and (C) perfusion bioreactor,

FIGS. 7 (A) and (B) are histograms illustrating the alkaloid content of the resin for the different bioreactor cultures. Dense right slanted dash=sanguinarine, sparse left slanted dash=chelerythrine, light gray=chelerubine, white=chelilutine, dark gray=macarpine. (A) Bioreactor with suspended resin; and (B) perfusion bioreactor,

FIGS. 8 (A), (B), (C) and (D) are graphs illustrating the cells nutritional behavior and intracellular status for the different bioreactor cultures. (A) Cells specific oxygen uptake rate with culture time; (B) Glucose concentration in the culture medium with culture time; (C) Intracellular concentration in nitrate with culture time; (D) Intracellular concentration in inorganic phosphate with culture time. (□) bioreactor culture without extractive phase; (Δ) bioreactor culture with free resin; () perfusion bioreactor culture with antifoam; (▾V) perfusion bioreactor culture without antifoam. Elicitation: time of elicitation with the chitin solution,

FIG. 9 is a graph illustrating the effect of growth conditions (batch culture, perfusion culture (exponential feed) and perfusion culture (calculated feed)) on E. californica cells,

FIG. 10 is a graph illustrating the comparison of sedimentation rates for E. californica and N. tabacum cells for a complete range of SCVs in a perfusion bioreactor,

FIG. 11 is an histogram representing continuous in-situ extraction of recombinant aprotinin from alfalfa suspension cells cultured in the perfusion bioreactor. Each bar represents an affinity column content in aprotinin. The time indicated corresponds to the time when each affinity column was harvested under medium perfusion condition, and;

FIG. 12 is a picture of a Western Blot of the samples eluted from the extraction columns using an anti-6His antibody. (In-situ extraction time for each column is indicated between brackets).

### DETAILED DESCRIPTION OF THE INVENTION

### Definitions

Unless specifically defined, the terms found in the present application have the meanings that one of skill in the art would normally attribute to them.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or method steps.

The term “about” is used to indicate that a value includes an inherent variation of error for the device or the method being employed to determine the value.

The term “alkaloid” as used herein is understood as being a substance defining any basic, organic, nitrogenous compound not only occurring naturally in an organism, but also their synthetic and semi-synthetic analogues and derivatives. Thus, as used herein, the term alkaloid covers not only naturally-occurring basic, organic, nitrogenous compounds but also derivatives and analogues thereof which are not naturally occurring and which may be neither basic nor nitrogenous. Most known alkaloids are phytochemicals, present as secondary metabolites in plant tissues (where they may play a role in defense), but some occur as secondary metabolites in the tissues of animals, microorganisms and fungi.

The term “derivative” as used herein is understood as being a substance which comprises the same basic carbon skeleton and carbon functionality in its structure as a given compound, but can also bear one or more substituents or rings.

The term “analogue” as used herein is understood as being a substance similar in structure to another compound but differing in some slight structural detail.

The abbreviation “DW” as used herein means dry cell weight.

The abbreviation “FW” or “WW” as used herein means fresh cell weight or wet weight.

The term “perfusion bioreactor” as used herein means a fluidized-bed reactor for cell culture designed for continuous operation as a perfusion system, i.e., a system in which fresh medium is fed to the bioreactor at the same rate as spent medium is removed.

The term “metabolite” or “metabolites” as used herein designates compounds that are naturally produced by an organism (such as a plant or animal) and that are directly involved in the normal growth, development or reproduction of the organism. This includes, but is not limited to, any compound produced by plant or animal cells, or genetically modified plant or animal cells, such as proteins, proteins or other types of chemical compounds.

The term “secondary metabolite” or “secondary metabolites” as used herein designates compounds that are naturally produced by an organism (such as a plant or animal) but that are not directly involved in the normal growth, development or reproduction of the organism. It is in this sense that they are “secondary”. The function or importance of these compounds to the organism includes the following: (1) use as a defense against predators, parasites and diseases, (2) use for interspecies competition, and (3) use to facilitate the reproductive processes (coloring agents, attractive smells, etc). This includes, but is not limited to, any compound produced by plant or animal cells, or genetically modified plant or animal cells, such as proteins, recombinant proteins and other types of chemical compounds. Examples of secondary metabolites include antibiotics and pigments.

The abbreviation “SCV” as used herein means sedimented cell volume.

The term “time of elicitation” as used herein means the time at which the eliciting agent is added to the culture. In the context of the experiments specified herein, the eliciting agent was a chitin extract prepared as described.

As used herein the term “cellular medium” means any liquid in which the cell may either grow, produce a desired product, or in which they can be kept. The term “cellular medium” may comprise for example, a cell culture medium, a buffer (e.g., phosphate buffer saline, etc.).

As used herein the term “separating means” is to be understood as a resin or matrix which allow separation of molecules from another or for separation of a desired product from undesired components. Such “separating means” may include without limitation, matrix for size exclusion chromatography, ion-exchange matrix, etc.

As used herein the term “capturing means” is to be understood herein as a resin or matrix which allow binding (e.g., selective binding) of desired products. Such “capturing means” may include, without limitation, affinity matrix.

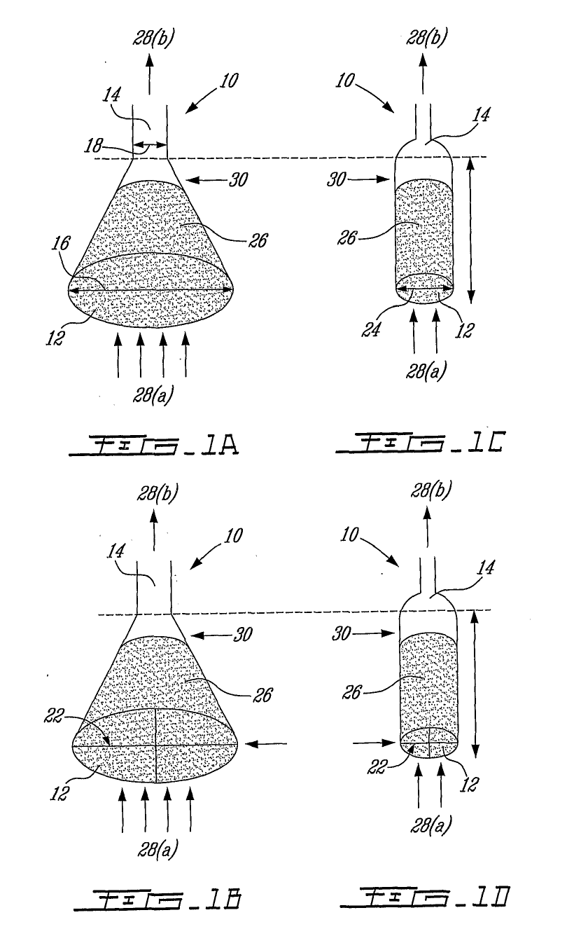

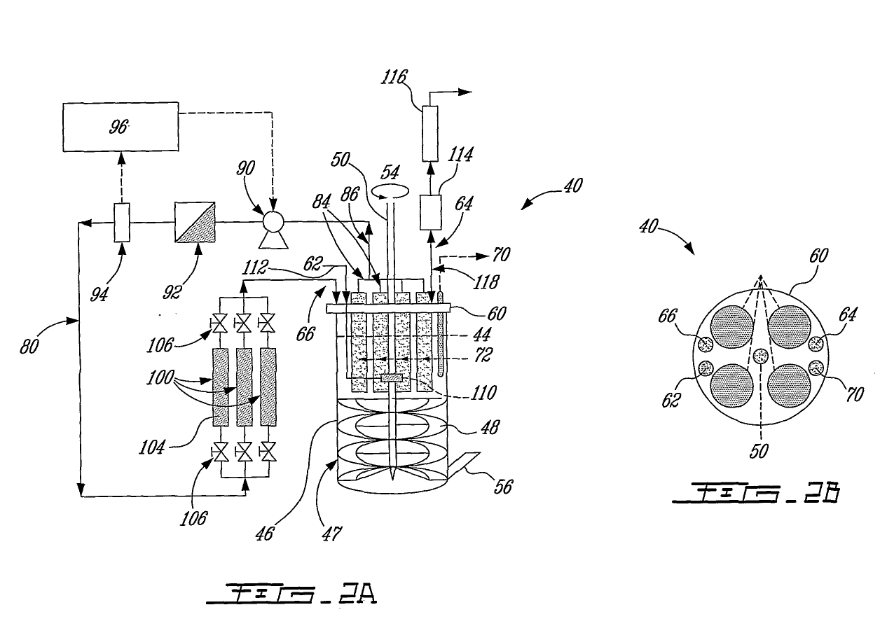

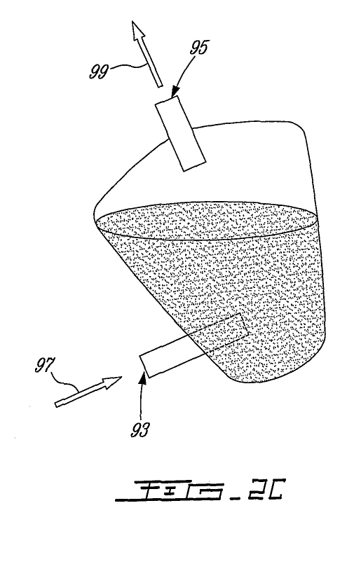

Non-restrictive illustrative embodiments of the sedimentation column will now be described in connection to FIG. 1 of the appended drawings.

Referring to FIGS. 1(a) to (d) of the appended drawings, non-restrictive illustrative embodiments of the sedimentation columns are generally identified by the reference (10). The sedimentation column (10) comprises an inlet opening (12) and an outlet opening (14).

FIG. 1(a) and FIG. 1(b) illustrate the funnel type sedimentation column, where the inlet opening diameter (16) is larger than the outlet opening diameter (18). In FIG. 1(b) the exemplary embodiment of the sedimentation column is illustrated with a vortex reducer (22) at the inlet opening (12), whereas in another exemplary embodiment, the sedimentation column illustrated in FIG. 1(a) the sedimentation column (10) does not have a vortex reducer.

FIG. 1(c) and FIG. 1(d) illustrate the cylindrical type sedimentation column having a defined internal diameter (24). In FIG. 1(d) the exemplary embodiment of the sedimentation column is illustrated with a vortex reducer (22) at the inlet opening (12), whereas in another exemplary embodiment of the sedimentation column illustrated in FIG. 1(c) the sedimentation column (10) do not have a vortex reducer. In exemplary embodiments of the invention, the internal diameter (24) of the cylindrical type of column may vary according to the needs of the user.

In accordance with exemplary embodiments of the present invention, cylindrical type sedimentation columns may have an internal diameter varying from about 20 to 50 mm for bioreactor systems of about 1 L to 3 L. However, the sedimentation column diameter may be selected according to the vessel's volume capacity. The length of each sedimentation column may vary to accommodate the bioreactor vessel size, agitation speed, configuration of the vortex reducer and cell species. The column may preferably long enough to allow a stable cell front bed to be established.

Although the vortex reducer (22) (FIGS. 1(b) and 1(d)) is illustrated as having a cross shape, the configuration of the vortex reducer may have other configurations such as a 3, 4, 5, etc.—branches star shape, a grid, etc. Other vortex may be used without departing from the scope of the invention.

In FIGS. 1(a) to (d), the flow of liquid (cell culture medium, or other appropriate media) (26), which circulates when the sedimentation columns are in use, is illustrated by arrows (28a and 28b). Arrows (28a) illustrate the entry of liquid (26) through the inlet opening (12) and into the sedimentation column (10) and arrow (28b) illustrate the exit of the liquid (26) from the sedimentation column (10). Using the parameters described herein, a stable cell front bed (a stable sedimentation front) may be obtained (30).

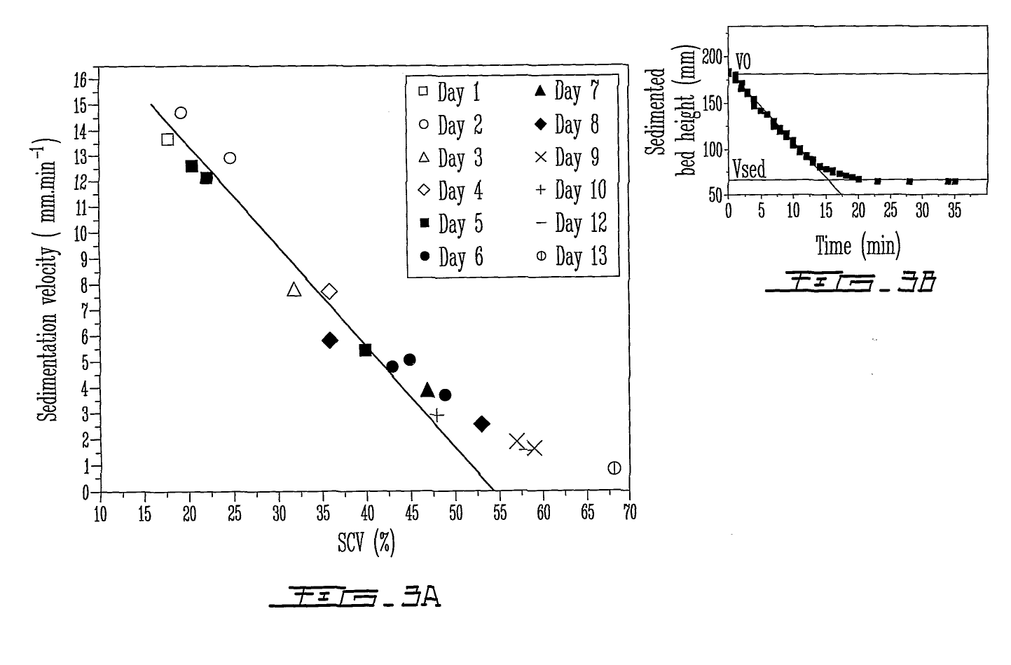

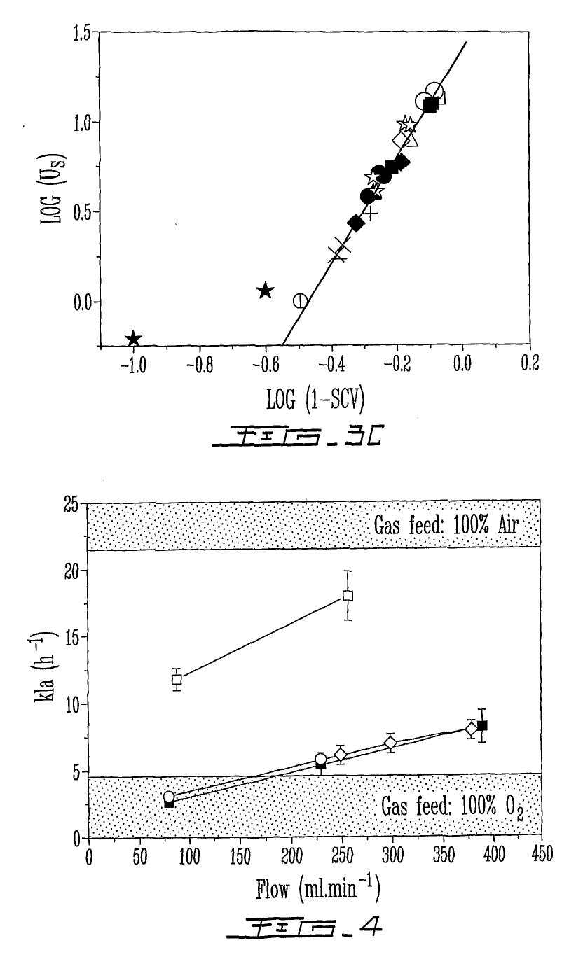

Non-restrictive illustrative embodiments of the perfusion bioreactor system will now be described in connection to FIGS. 2(a) and 2(b) of the appended drawings.

Referring to FIGS. 2(a) and (b) of the appended drawings, non-restrictive illustrative embodiments of the perfusion bioreactor system is generally identified by reference (40). The perfusion bioreactor system of FIGS. 2(a) and 2(b) are illustrated without a cellular biomass. However, the liquid level is illustrated by the dashed line (44).

The perfusion bioreactor system (40) comprises a vessel (46) in which a cellular biomass may be cultured. The apparatus is provided with an agitator assembly, generally identified as (47) in FIG. 2(a). The agitator assembly (47) may comprise for example, an impeller (48) which is found at the bottom of the vessel (46) and an agitator shaft (50) connected to the impeller (48). The impeller (48) may be actuated in a clockwise direction or in a counterclockwise direction (54). The agitator shaft (50) is actuated by a motor (not illustrated). The impeller (48) have a helical shape, in the present case; a double-helical ribbon impeller. The vessel (46) is also provided with a sampling port (56) allowing the removal of samples for analytical purposes during cell growth and/or for assessment of production of a desired product.

The vessel (46) is closed by a cover or head plate (60) which is preferably sealable. The cover (60) may allows the passage of gas entry means (62), gas exit means (64), cell culture medium entry means (66), gas probes (70) (e.g., an oxygen probe) and sedimentation columns (72) as well as passage for the agitator shaft (50). The exemplary embodiment of FIGS. 2(a) and 2(b) are presented with four sedimentation columns (72). However, as indicated herein, the number of columns may vary.

The bioreactor system (40) is provided with an extractor assembly generally defined by reference (80), which allows circulation of the cell culture medium, extraction of the desired product and, if desired, recirculation of the medium (substantially free of the product) into the vessel (46).

The extractor assembly (80) may comprise at least one sedimentation column defining a channel (81). However, the extractor assembly (80) may comprise more than one sedimentation column (72). The number of sedimentation column may be selected based on the need of the user and depending on the volume of the bioreactor. The channel may be so configured as to provide for a separation of the cellular medium and the cells when the cellular biomass enters the channel.

In accordance with the present invention the extractor assembly may comprise, at least two sedimentation columns, at least three sedimentations columns, at least four sedimentation columns, etc. The sedimentation column may preferably comprise a vortex reducer. The configuration of the vortex reducer however, is not intended to be limited to a specific shape.

The sedimentation columns (72) may preferably be installed along a substantially vertical axis of the vessel (46) so that the inlet opening is substantially parallel to the cell culture medium surface (44), i.e., the liquid surface. Tubing (84) exiting the outlet opening of the sedimentation columns (72) are joined into a single tubing (86) and represent means for allowing cell culture medium exit. A pump (90) (e.g., peristaltic pump) allows circulation of the cell culture medium out of the bioreactor system (40). A pressure sensor (94) and over-pressure detector (96) are also provided.

The extractor assembly (80) may also optionally comprise a second sedimentation module (92). The second sedimentation module (92) is installed near the outlet end of the sedimentation column. An exemplary embodiment of the second sedimentation module (92) is illustrated in FIG. 2(c).

The second sedimentation module (92) is added to achieve an efficient clarification of medium from cells and cell debris. The second sedimentation module (92) has an overall conical shape with the smaller section positioned at the bottom and the larger section at the top. In use, the medium enters the inlet (93) and circulates from the bottom to the top of the module and exit the second sedimentation module through the outlet (95). Setting the module with an angle of 45° along a vertical axis assures a best efficiency. As such, cells and cell debris are allowed to sediment at the bottom. The flow of cellular medium is illustrated by arrows (97) and (99).

The extractor assembly (80) may also optionally comprise at least one extraction column. (100) The extraction column (100) may be advantageously added to the bioreactor apparatus for selectively removing cellular products comprised within the cell culture media. More than one extraction column may be mounted on the bioreactor apparatus. These extraction columns may be in simultaneous use or in sequential used.

The extraction columns may also be advantageously kept at a desired temperature (e.g., 4° C.). This characteristic is especially useful for product which are temperature-sensitive.

Extraction columns (100) are thus installed at the end of the extractor assembly (80). These extraction columns (100) contains fluidized resins (104) and are provided with valves (106) which, when in an opened position, allow circulation of cell culture medium through a desired extraction column (100) and thus allow extraction of the desired product from the cell culture medium.

Extraction columns (100) of varying capacity (e.g., internal diameter, length) are made available and are selected depending on the extraction velocity and/or the system pressure.

The perfusion bioreactor system (40) is also provided with gas entry means (62) and gas exit means (64).

The gas entry means (62) may be provided with a gas diffuser (110) connected to the bioreactor system (40) through appropriate tubings (112).

The gas exit means (64) may be provided with a foam trap (114) and a condenser (116) which may be connected to the bioreactor system (40) through appropriate tubings (118).

### Materials and Methods

### Plant Cell Culture

Eschscholtzia californica cell suspension cultures were maintained in B5 liquid medium (Gamborg et al., 1968) containing 30 g.l⁻¹ glucose (Sigma-Aldrich, Oakville, Ontario, Canada; cat.#: G5767), 0.2 mg.l⁻¹ of 2,4 dichlorophenoxyacetic acid (Sigma-Aldrich; cat.#: D7299) and 0.1 mg.l⁻¹ kinetin (Sigma-Aldrich; cat.#: K0753). Medium pH was adjusted to 5.6 using 1 M KOH before sterilization (121° C., 1 atm, 25 min). The suspension (80 g) was then transferred into a 500 ml Erlenmeyer flask containing fresh medium (170 g). The 250 ml suspension cultures were subcultured every 10-11 days, when the sedimented cell volume reached 70-80% of the total volume after 5 minutes without flask agitation. Cultures were maintained at 130 rpm, 25±3° C., under normal continuous laboratory light.

### Basic Bioreactor Configuration

Control cultures were performed using the bioreactor without an external loop as described in this section. The bioreactor with an external loop used in the perfusion cultures is described in the next section and it should be noted that the liquid recirculation was started at the time of elicitation. A 3-l (2.5-l working volume) in-house bioreactor composed of 316-L stainless steel (SS) parts and a glass vessel (12.5 cm×27 cm, VWR, Montreal, Quebec, Canada; cat.#: 36390.086), and having the same geometrical ratios as Jolicoeur et al. (1992) was used (FIG. 1). A double helical-ribbon impeller (120 mm height×115 mm O.D., 22 mm width) was used. However, other types such a marine impeller, anchor impeller or Rushton impeller, etc. could be used. Porous (2 μm) 316-L SS gas sparger which generated fine bubbles was used at the bottom or set at 6 cm from the surface of the liquid. Dissolved oxygen measurement was performed by a polarographic probe (Mettler Toledo, Mississauga, Ontario, Canada; cat.#: InPro 6800) connected to a data acquisition system (Virgo, Longueuil, Quebec, Canada). The probe was positioned at 10 cm below the liquid surface. The k_{L}a values of the bioreactors were measured in triplicate with water by the gassing (air) method. Degassing was performed using an N₂ gas fed at the same flow rate as air. Different conditions of aeration and agitation were tested to study the transfer in the bioreactor filled with water. Agitation was tested at 45 rpm and 60 rpm in clockwise (upward pumping) and counterclockwise (downward pumping) rotation. The gas sparger was located at the bottom for all bioreactor cultures, except for the perfusion culture where the sparger was set at 6 cm below the liquid surface. The bioreactors were sterilized in an autoclave (121° C., 1 atm, 90 min) with the fresh medium (the same composition as for flasks). The wet resins and the elicited solution (see below) were sterilized in a flask together with the bioreactor.

### Design of the Perfusion Bioreactor

Studies performed on cells sedimentation velocity and on the separation devices were performed under non aseptic and non elicited conditions using fresh cell suspensions obtained from shake flask cultures. Successive experiments were carried out with a given suspension for a maximum period of one day to avoid bias from contamination.

### Sedimentation Velocity of Plant Cell Culture

The sedimentation velocity was determined measuring the velocity at which the sedimentation front evolved at steady-state (FIGS. 1B and 2B). The sedimentation front was defined as the cell bed suspension/cell-free liquid interface. A 100-ml cell suspension taken from shake flask cultures was used and placed into a 28 mm I.D. glass tube closed at the bottom. The SCV was determined at a stable sedimentation front, which took from 20 to 60 min. The SCV (in %) was calculated as the ratio of the sedimented cell volume V_{sed} divided by the initial suspension volume V₀ of 100 ml.

### Sedimentation Device Configuration

Four cell/medium glass separation devices were tested for their separation efficacy. Device of type A consisted of a funnel (25 mm×38 mm I.D.×20 mm I.D.), and devices of type B, C and D were cylindrical columns of different diameters (B: 90 mm×26 mm I.D.; C: 90 mm×31 mm I.D.; D: 165 mm×41 mm I.D.). All devices were tested equipped with or without a cross at their inlet (FIG. 1). The cross was used to dampen the liquid vortexes and oscillations and they were made of 1 mm×10 mm height polystyrene sheet. The branches of the vortex reducer may preferably be thin. For example, a height of about 1 cm was shown to be optimal for a 2.5 L vessel. The devices A, B and C were immersed 3 cm in a cell suspension contained into a polypropylene vessel (165 mm×85 mm I.D.) equipped with a double helical ribbon impeller with geometrical ratios similar to that of the 2.5-l bioreactor. It was determined that agitation needed to be set at a maximum of 30 rpm (counterclockwise, downward pumping at the blades), otherwise the cell sedimentation front was unstable with time. The cell suspension initial volume was 650 ml and the liquid level was kept at the initial level by adding cell suspension when needed (e.g. when filling the recirculation loop). For device D, a larger polypropylene vessel of 145 mm×105 mm I.D. was used with an initial cell suspension volume of 1000 ml. The same impeller as for the smaller vessel (650 ml) was used and fixed at a rotational speed of 60 rpm, upward or downward pumping as specified. The liquid was pumped (peristaltic pump; Masterflex, Labcor, Anjou, Quebec, Canada; cat.#: A77521) from the outlet of the device and recirculated into the vessel. Liquid at the outlet of the device was sampled and centrifuged for 5 min (16 000 g) and the fresh cell weight (WW) of the residue was measured, representing the loss in cells and debris.

### Extraction Column Configuration

Extraction columns containing polymeric adsorbent resin, such as XAD-7, were designed to allow for continuous long term liquid flow. Compact bed configuration showed a high susceptibility to clogging caused by cell debris accumulation. A fluidized bed (upward liquid flow) was then selected because it may enable the cell debris to pass freely through the fluidized bed of resins. A 45 μm stainless steel mesh (Spectrum Laboratories, Rancho Dominguez, Calif., USA) was installed at the column top end to avoid resin entrainment with liquid flow. The fluidization velocity of the XAD-7 resin was determined experimentally at 70 mm.min⁻¹ in culture medium. Since, the liquid flow rate is set from the conditions required in the sedimentation devices, to maintain a stable sedimentation front, a series of extraction columns with different diameters enabling resins fluidization (liquid upward velocity ˜70 mm.min⁻¹) were designed to be used successively along a culture. Each column diameter was determined using SCV data for the first culture series without the recirculation loop. In one embodiment of the invention, shown in FIG. 2, the following three columns were found to be efficient: column I (26 mm I.D.) for days 4, 5 et 6, column II (17 mm I.D.) for days 7 et 8 and column III (13 mm I.D.) from day 9. It should be understood, however, that the type and number of extraction columns may be varied, and that consequently the invention is not restricted to the use of the type and number of columns exemplified herein. For example, any resin that is known to adsorb secondary metabolites, including, but not limited to, the XAD-7 used here and other members of the XAD family of resins, can be used in the columns.

Bioreactor Cultures

The agitation was set at 60 rpm (clockwise, upward pumping) for cultures without recirculation loop. The perfusion cultures were performed at 40 rpm (counterclockwise). During cultures, dissolved oxygen was maintained by a control system (Virgo) at a minimum of 60% air saturation in the medium by mixing air and O₂ with 2 mass flow controllers (Tylan, Mykrolis, Billerica, Mass., USA; cat.#: FC 260). During cultivation, the cell oxygen demand was such that air was initially injected at a maximum flow rate of 200 ml.min⁻¹. O₂ was then added while keeping the total flow rate constant at 200 ml.min⁻¹ until a ratio of 50/50 was reached. Then, to avoid high oxygen concentration, total gas flow rate was increased up to 400 ml.min⁻¹. The temperature was maintained at 25±2° C. and the culture was performed in the dark to avoid alkaloid degradation by light.

Three series of bioreactor cultures were performed. A first series consisted of two bioreactors running in parallel, one with extraction XAD-7 resin (50 g.l⁻¹) added to the cell suspension at the time of elicitation and one without resin. In a second series, a perfusion bioreactor (the resin content of the extraction columns has been described previously) was ran in parallel to a bioreactor with suspended resins (i.e., free resins). Finally, the perfusion bioreactor culture was repeated in a third series of cultures.

The working volume was 2.5 l for the basic bioreactor and 2.34 l for the perfusion bioreactor before elicitation, with the four separation devices (and the recirculation loop) maintained suspension-free (i.e., empty). All bioreactors were inoculated with 11 days old cell suspensions obtained from shake flasks, at a ratio of 33% (v/v), which resulted in an average initial biomass concentration of 3.7 gDW.l⁻¹. The inoculation ratio was 47% (v/v) and the initial concentration 5.1 gDW.l⁻¹ for the perfusion bioreactor. Different volumes of inocula or initial biomass concentrations were used in such a way that similar cell concentrations were reached at elicitation (day 4+) in all bioreactor cultures. All bioreactor cultures were thus elicited at day 4 by the addition of a chitin solution (see below). Other elicitor may be used without departing from the scope of the invention, for example, a second messenger involved in stress signal may efficiently be used. Other specific examples of elicitors include for example, jasmonic acid, salicylic acid, yeast extract.

The elicitation solution was pumped into the cell suspension for a final concentration of 160 ml.l⁻¹. For the perfusion reactor, fresh medium (690 ml) and chitin solution (540 ml in medium) were added to fill the bioreactor volume and the recirculation loop (860 ml) for a total working volume of 3.2 l.

The inlet ends of the sedimentation columns were 7 cm below the liquid medium surface. At elicitation (day 4+), the medium was continuously pumped (tubing of 1.6 mm I.D., one pump head per separation device, peristaltic pump, Masterflex; cat.#: 77390-00) at the outlet of the four separation devices (device D with a 316-L SS cross of 10 mm height). The four tubes were connected together after the pump heads to a common tube (tubing of 6 mm internal diameter (I.D.)). The medium then flew to the second stage sedimentation module then to extraction columns for finally being recirculated into the bioreactor to the cell suspension. The second stage sedimentation module was designed to retain the few cells and cell debris that are leaving the sedimentation columns and which can affect fluidization of the adsorption beads and could colonize onto the grid supporting the extraction beads. This module retained less than one ml of cells for the culture duration.

The cell suspension was pumped at 45 ml.min⁻¹ on day 4, 5 and 6 to the column I (185 mm×26 mm, I.D.; Nalgene) containing 29.8 g XAD-7 resins. On days 7 and 8 the pumping rate was decreased to 16 ml.min⁻¹ and column I was changed manually for column II (240 mm×17 mm I.D.) containing 23.6 g resin. On day 9, the pumping rate was decreased to 11 ml.min⁻¹ and column II was changed for column III (330 mm×13 mm I.D.) containing 17.4 g resin. The rationale for this column change was described in the Extraction column configuration section, above. A pressure probe (Dynisco, North York, Ontario, Canada) was used to measure the pressure at the input of the resin columns. When an increase of pressure of 0.5 atm was observed, the recirculation was automatically interrupted. A foam trap was installed before the condenser in order to retain the cells entrained by the outlet air flow. The trapped foam was regularly reinjected in the bioreactor. A 1 ml volume of anti-foam (Mazu, BASF Corporation, Gurnee, Ill., USA) was injected at elicitation in the perfusion bioreactor of the second series of cultures. Because of the low production levels observed in that culture no anti-foam was added in the perfusion bioreactor of the third series of cultures.

### Adsorbent Resin Preparation

The neutral polymeric XAD-7 Amberlite resins were used for the adsorption of the alkaloids in the extraction columns. The resins (Sigma-Aldrich, cat.#: XAD7) were prepared as follow. Resins were soaked in methanol for a minimum of 24 h and then washed four times in deionized water to remove all traces of methanol. After separation on a nylon mesh (400 μm), large resin fraction (>400 μm) was kept for the experiments and stored in deionized water.

### Elicitation Solution Preparation

A 10 g.l⁻¹ crude chitin solution was prepared by extraction, crushing crude chitin (Sigma-Aldrich; cat.#: C3387) in deionized water in a mortar. The mixture was then autoclaved at 121° C. for 30 minutes, stirred during cool down and filtered under vacuum on two layers of Miracloth filter membrane (Calbiochem, La Jolla, Calif., USA; cat.#: 475855). This solution was then used to prepare B5 medium used at elicitation.

### Analytical

Cell suspensions were sampled from the bioreactors (30-50 ml) on a daily basis and analyzed for pH, fresh and dry cell weights, extracellular and intracellular nutrients (sugars, anions, cations), and for the alkaloid contents of cells, medium and resins. The fresh cell weight (FW or WW) was obtained by filtration of 10-ml cell suspension through a Whatman paper filter (Fisher Scientific Pittsburgh, Pa., USA; cat.#: 09874-48) under vacuum. The wet cell sample was then placed at 60° C. in an oven for 24 hours and the dry cell weight was measured. The remaining cell suspension sample was used for subsequent analysis and processed as follows. After filtration through a Whatman GF/D 47-mm filter (Fisher Scientific) under light vacuum, medium was kept at −20° C. and cells were washed twice with 30 ml distilled water. Then the cells were immediately frozen in liquid nitrogen, crushed in a pre-cooled mortar, aliquoted in 2-ml Cryovials® (Fisher Scientific, Nepean, Ontario, Canada, cat #03-374-21) then stored in liquid nitrogen until analysis. Before analysis, frozen cells were freeze-dried overnight under vacuum (Dura-Top FTS Systems, Bulk Tray Dryer, New York, USA) for 24 hours at −5° C. and −97 kPa for subsequent measurements of intracellular compounds.

### Sugars

10 mg of lyophilized biomass was washed three times with 80% ethanol followed each time by a 15-min sonication. The supernatant was centrifuged 5 minutes at 16 000 g and analyzed by HPLC (see below). The culture medium was filtered at 0.45 μm (Fisher Scientific; cat.#: 09-902-10) and analyzed by HPLC as follows. The contents in glucose, fructose and sucrose were determined using a Beckman Coulter™ HPLC system (pump module 126, auto-sampler model 508) and a refractive index detector (ERC-7515-A). An Alltech Prevail™ carbohydrate ES analytical column (250 mm×4.6 mm I.D., 5 μm) was coupled with an Alltech Prevail™ carbohydrate ES guard column (7.5 mm×4.6 mm I.D., 5 μm) and maintained at 35° C. The mobile phase consisted of a solvent mix 75:25 (v/v) acetonitrile and water at a flow rate of 1.0 ml.min⁻¹. The injection volume was 20 μL.

### Ions

10 mg of lyophilized biomass was washed with 1.5 ml of Trichloroacetic acid (5%; w/v). The solution was sonicated at 40° C. for 30 minutes and then centrifuged 10 minutes at 16 000 g. The supernatant was analyzed by HPLC. The culture medium was filtered at 0.45 μm (Fisher Scientific; cat.#: 09-902-10) and analyzed by HPLC. Contents in major ions (Cl⁻, NO₃⁻, PO₄²⁻, SO₄²⁻, NH₄⁺, K⁺, Na⁺, Mg²⁺, Ca²⁺) were determined using a Dionex IC system (AI-450) equipped with a gradient pump module, a pulsed electrochemical detector in mode conductivity, and a ThermoFinnigan Autosampler AS3500. For the anions, the separation was performed with a Dionex IonPac® AS-14 analytical column (250 mm×4 mm I.D., 9 μm) coupled with a Dionex IonPac® AG-14 guard column (50 mm×4 mm I.D., 9 μm) and a ASRS® Ultra (4 mm) suppressor. The mobile phase consisted of a 2 mM Na₂CO₃/1 mM NaHCO₃ solution at a rate of 1.0 ml.min⁻¹. For the cations, an IonPac® CS-12A analytical column (250 mm×4 mm I.D., 8 μm), an IonPac® CG-12A guard column (50 mm×4 mm I.D., 8 μm) and a CSRS® Ultra II (4 mm) suppressor were used. The mobile phase was 20 mM methanesulphonic acid solution at a rate of 0.9 ml.min⁻¹.

### Alkaloids

40 mg of lyophilized biomass was extracted three times with 1.5 ml acidified MeOH (0.5% HCl, v/v) followed each time by a 15-min sonication. The supernatant was centrifuged 10 minutes at 16 000 g, evaporated under vacuum and suspended in 400 μl acidified MeOH (0.5% HCl, v/v) for HPLC analyses. Alkaloids extraction of extracellular medium was performed by solid phase extraction on a Phenomenex Strata™ C₁₈-E column (3 ml capacity, 300 mg packing). The conditioning of the column was done with 3 ml acidified MeOH (0.5% HCl; v/v) followed by 3 ml distilled water. The medium (10 ml) was loaded and 10% (v/v) acidified MeOH in water (3 ml) was applied for washing. The final elution was done with 1 ml of acidified MeOH (0.5% HCl; v/v). The solution was filtered (0.45 μm, PTFE membrane, VWR; cat.#: 28143-981) before analysis by HPLC. After separation by sedimentation from biomass, the resins were rinsed in distilled water. Total resin content in the sample was extracted 5 times in 10 ml acidified MeOH (0.5% HCl; v/v). The volume was adjusted at 50 ml with acidified MeOH (0.5% HCl; v/v). To analyze the content in alkaloids of an extractive column with resin, the total resin content was extracted 5 times with 60 ml acidified MeOH (0.5% HCl; v/v). The volume was adjusted at 300 ml with acidified MeOH (0.5% HCl; v/v). The solution (1 ml) from each sample was filtered (0.45 μm, PTFE membrane, Gelman Laboratory) before HPLC analysis.

Extracts from cells, medium and resins were analyzed for alkaloid content using the following chromatographic method described previously (Klvana et al., 2004, 2005). The HPLC apparatus used consisted of a model 126 Beckman Coulter™ pump module and a model 508 Beckman Coulter™ auto-sampler, coupled with a model 821-FP Jasco® fluorescence detector and a model 168 Beckman Coulter™ photo diode array absorbance detector. Chromatographic separation was obtained using a Zorbax™ Eclipse XDB-C₁₈ column (250 mm×4.6 mm I.D.; 5 μm) coupled with a Securiguard C₁₈ guard column maintained at 35° C., with a flow rate of 1.5 mL.min⁻¹ and 20 μl injection volume. The mobile phase consisted of solvent A: 50 mM H₃PO₄, pH adjusted to 3 with KOH and solvent B: acetonitrile. The elution profile was: 0-2 min: 25% B; 2-12 min: linear gradient to 35% B; 12-14 min: 35% B; 14-22 min: linear gradient to 80% B; 22-29 min: 80% B; 29-31 min: linear gradient to 25% B, 31-33 min: 25%. Sanguinarine, chelerythrine and chelirubine were quantified by fluorescence (ex. 330 nm, em. 570 nm) while macarpine and chelilutine were quantified by absorbance at a wavelength of 341 nm. Peak purity was verified by UV spectra symmetry. Calibration curves were obtained with standards that were purchased: sanguinarine and chelerythrine (Sigma) and standards obtained from a semi-preparative method (Klvana et al., 2005).

### Example 1

### E. californica Cell Cultures without Medium Recirculation

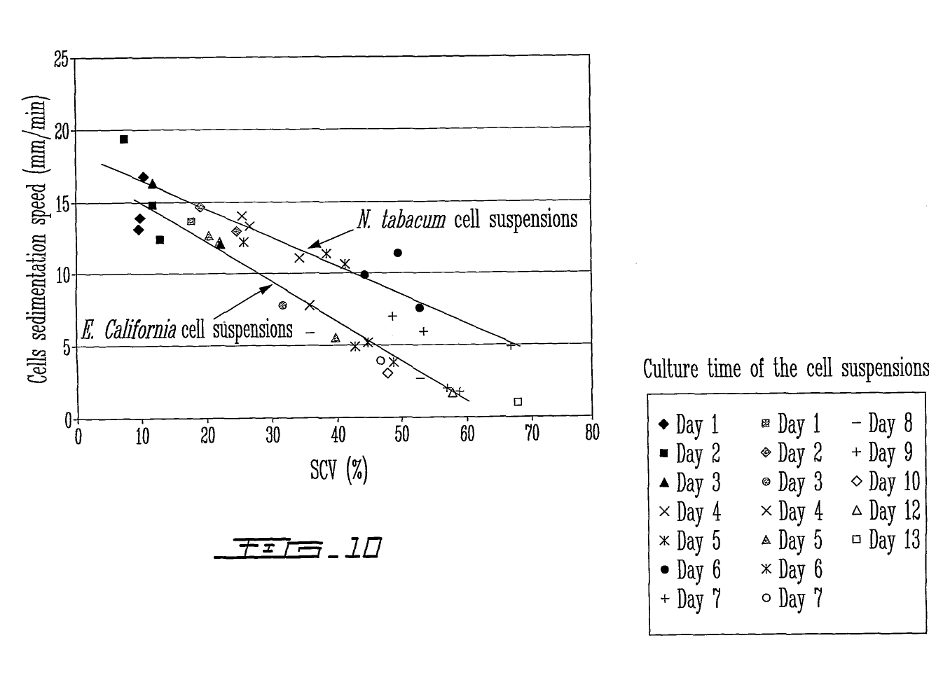

E. californica cells were cultured in the perfusion bioreactor with fresh medium feed, but without any medium recirculation. As shown in FIG. 9, it was confirmed that an exponential feed of fresh culture medium led to a maximum cell growth and a cell density which were sustained for 4 days (Δ), as compared to the batch culture with 100% medium recirculation (◯). The use of a feed, calculated from a model (★), led to a stable cell density (days 5-9), and then the use of an exponential feed allowed a maximal growth rate to be reached and a cell density which was close to the maximum theoretical value (340 gFW/L vs 365 gFW/L). The bioreactor shown was perfused for 20 days without any operational problems.

### Example 2

### Tobacco Suspension Cell Culture

Tobacco suspension cells exhibit a faster sedimentation rate for the complete range in SCVs (FIG. 10). This confirms that the bioreactor can be perfused at a higher rate for tobacco cells that for E. californica cells, the possible medium perfusion rate being close to the cells sedimentation rate. For example, the medium perfusion rate at a SCV of 60% will be close to 1 mm/min for E. californica and close to 6.5 for N. tabacum.

### Results

### Cell Sedimentation Velocity is Related to Cell Suspension SCV

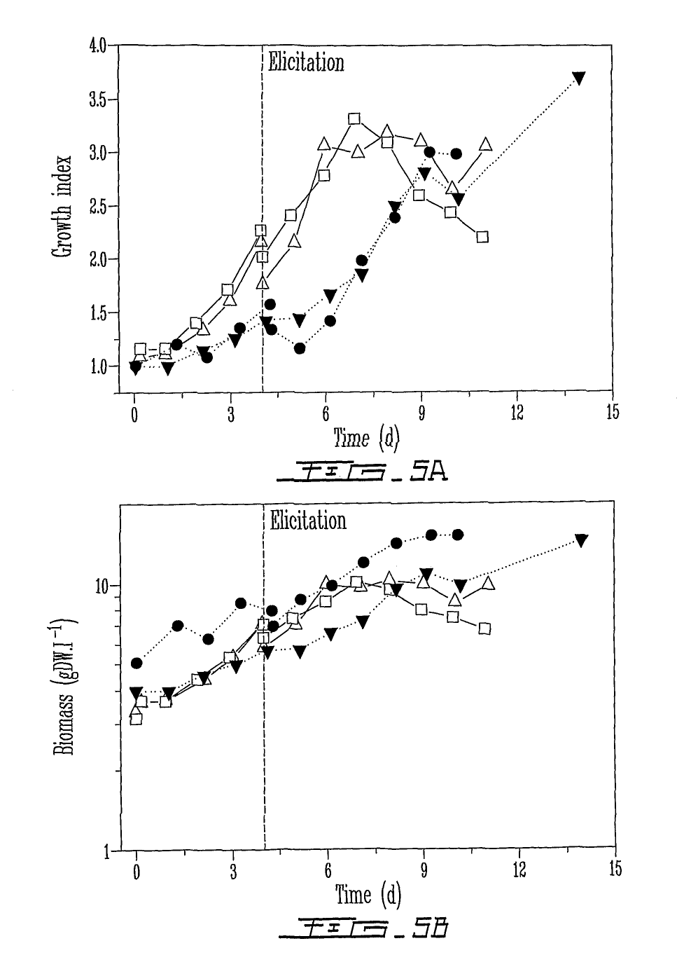

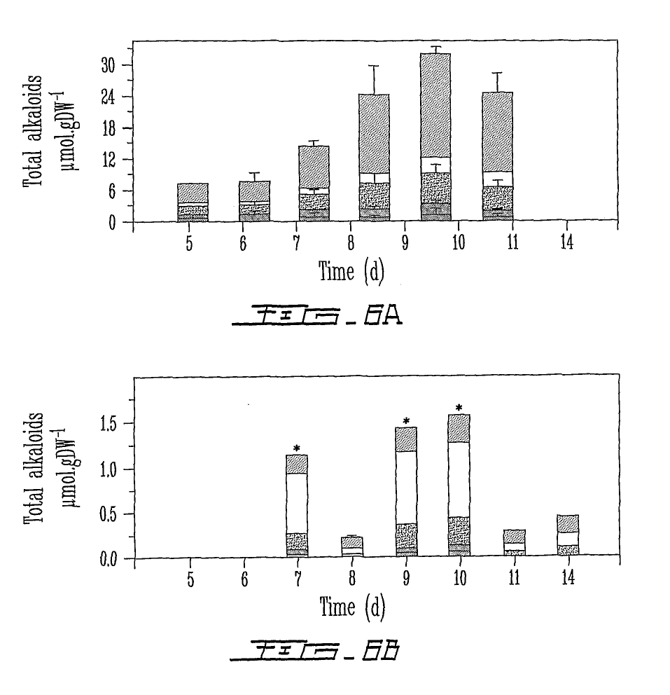

The cell/medium separation approach was based on sedimentation. Because it had been previously observed that the porosity of a cell bed evolves with culture age and may differ from subculturing (Gmati et al., 2004), the SCV was used rather than suspension DW or WW content to characterize the cell suspension. Determined in the linear region of the cell bed, height rate decreased with time (SD=0.7%, R²=99.6%) (FIG. 3), and the cell sedimentation velocity showed a linear decrease with the cell suspension SCV (FIG. 3A).

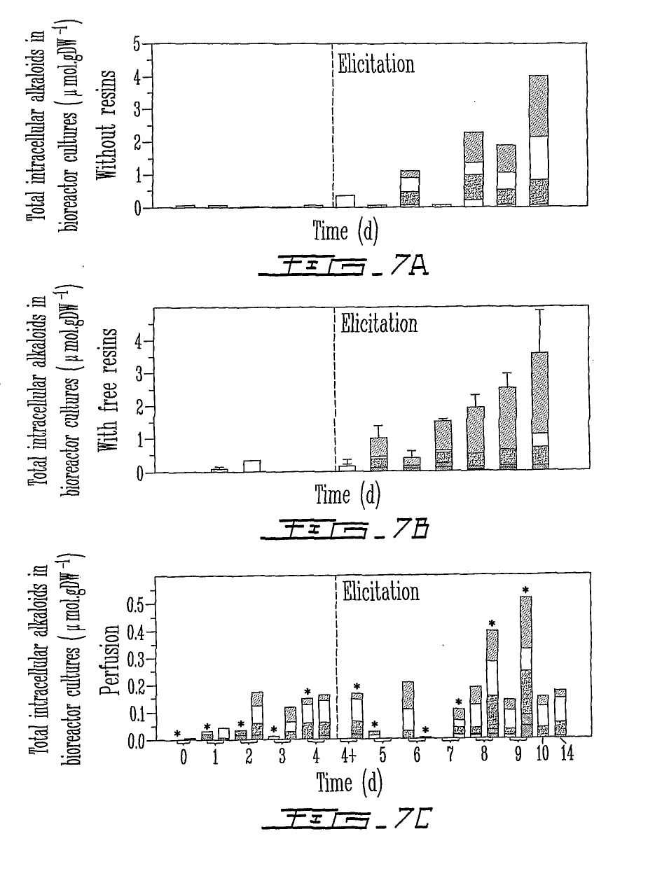

The suspension SCV was also preferred to the suspension packed cell volume (PCV) because SCV was directly obtained after measuring the cells sedimentation velocity and gives a picture of the cell suspension density. However, PCVs represented 81±2.9% of SCVs for Eschscholtzia californica cell suspension, a value which showed to be constant with suspension age between day 2 to day 10 (data not showed). Cell suspension PCV was obtained after centrifugation for 2 min at 2000 g of a cell suspension sample (Ryu et al., 1990). The cell sedimentation velocity (Uₛ) was determined in the linear region of the cell bed height decrease rate with time (SD=0.7%, R²=99.6%) (FIG. 4B). The cells sedimentation velocity showed to decrease linearly with the cell suspension SCV (FIG. 3A) and it is very interesting to observe that the sedimentation velocities obtained from the sedimentation experiments using shake flask samples and that calculated from perfusion experiments in model system and in bioreactor followed the same trend (results presented and discussed in the next section). The variations of Us as a function of SCV can be described by a modified Richardson-Zaki correlation law (Richardson and Zaki, 1954), often used to describe the hindered settling phenomena. This law is written as.

$${U}_{S} = {{U}_{T} [ 1 - \frac{C}{{C}_{S}} ]}^{n} = {{U}_{T} [ 1 - SCV ]}^{n}$$

where C and C_{S} are the cell volume fraction in the original suspension and the sedimented bed respectively. The ratio of C/C_{S} is equal to the ratio of V_{sed}/V₀=SCV. FIG. 4C shows the linear variation of U_{S} with SCV in log-log coordinates (R²=97.5%), resulting in a cell terminal velocity U_{T}=27.7 mm.min⁻¹ and a bed expansion index n=3.05.

However, at high cell density, other hydrodynamic and steric effects can affect cell sedimentation which generally results in deviation from the Richardson-Zaki correlation. This phenomenon was observed at SCV above 70% (or even above 55%) with a decreased in the sedimentation velocity (FIG. 3B) and where the observed values of U_{S} fall away from the Richardson-Zaki correlation. Results shown in FIG. 3 also suggest that suspension SCV is a more reliable parameter than DW and cell age to predict or to determine a suspension sedimentation velocity. These results therefore clearly show that the cell sedimentation velocity evolves with culture time. It is thus expected that the liquid pumping rate through cell/medium sedimentation devices will also have to be changed with time as discussed in the following sections.

### Screening of Efficient Separation Device Design

Two designs for the separation device, such as conical (a funnel, type A) and cylindrical (types B, C and D), were studied with cell suspensions in an agitated vessel with geometries similar to that of the bioreactor used for the cultures (see Materials and Methods). The effects of liquid pumping velocity and of device geometry and size on the establishment of a stable cell/medium separation front were characterized.

The separation efficiency of the columns was monitored measuring the cells and cell debris wet weight at the outlet of the columns. Then, since this wet material may differ from the composition of a cell suspension it was decided to use and compare wet weight (WW) contents of the inlet cell suspension and the outlet from the separation columns.

The type A device showed a high sensitivity to hydrodynamics and was rejected. Immerged into a cell suspension containing 240 gWW.l⁻¹ (55% SCV, 1.5 mm.min⁻¹ sedimented velocity), the outlet flow at steady-state contained 40 gWW.l⁻¹ pumping at 1 ml.min⁻¹ (0.9 mm.min⁻¹ pumping velocity at inlet). The addition of a cross at the inlet of the device resulted in reduction in the disturbances induced by the reactor impeller but without enabling the establishment of a stable cell/medium separation front at any pumping rate (data not shown). Cell concentration in the outlet flow at steady-state was reduced to 10 g WW.l⁻¹ at 1 ml.min⁻¹ (0.9 mm.min⁻¹ pumping velocity).

The Type B cylindrical device improved cell separation, with an outlet flow of 6 gWW.l⁻¹ at a flow rate of 1 ml.min⁻¹ (1.9 mm.min⁻¹ pumping velocity) for a similar cell suspension (240 gWW.l⁻¹, 1.5 mm.min⁻¹ sedimented velocity). Increasing column diameter to 31 mm (Type C device) increased flow rate capacity with stable front conditions. Three cell suspensions with sedimentation velocities of 5.5 mm.min⁻¹, 4.8 mm.min⁻¹ and 2.0 mm.min⁻¹ were used and the liquid flow rate was adjusted to impose liquid velocities equal to the respective sedimentation velocities of 4.1 ml.min⁻¹, 3.6 ml.min⁻¹, 1.5 ml.min⁻¹, respectively. The cell concentration in the outlet flow was then 4±2 gWW.l⁻¹ for all conditions. At higher flow rates (5.6 ml.min⁻¹ or 7.4 mm.min⁻¹, 5 ml.min⁻¹ or 6.6 mm.min⁻¹, 3.1 ml.min⁻¹ or 4.1 mm.min⁻¹) the front never reached stability.

These results suggest that pumping rate (liquid velocity or extraction rate) should preferably be slightly lower than the cells' sedimentation velocity in order to have a stable separation front, because of the steric and hydrodynamic effects suggested previously. The flow rate required to obtain a stable front varied linearly with column diameters from 26, 31 and 41 mm, suggesting that there was no wall effect at the scale of this study.

Type D column was challenged with high density cell suspensions obtained after removing liquid medium over sedimented cell bed of 5-d-old cells cultured in shake flask. Suspensions at 75% SCV (60% PCV) and 90% SCV (70% PCV) were then obtained and placed into the perfusion model system. Stable fronts were obtained at liquid pumping velocities of 1.14 and 0.62 mm.min⁻¹ into the sedimentation column, respectively for cell suspensions at 75% and 90% SCV (FIG. 3(A)).

### The Use of Separation Devices Required Bioreactor Design Modifications

### Position of the Separation Device

The presence of separation devices at the head plate of a 2.5-l bioreactor has to allow for the installation of many other essential elements such as the dissolved oxygen probe, gas inlet and outlet lines, liquid inlet line and a mechanical seal. In a sample embodiment, a set of four sedimentation columns with a larger diameter (type D: 165 mm×41 mm I.D.) was therefore selected and used to replace the usual surface baffles (FIG. 2) that function to break the liquid vortex (Jolicoeur et al., 1992). It should be understood, however, that the type and number of sedimentation columns may be varied, and that consequently the invention is not restricted to the use of the type and number of columns exemplified herein.

Because of the geometrical constraints of the bioreactor setup (i.e., the free space over the helical impellers), immersion of the separation device was limited to 8 cm below the liquid surface. The influence of the distance between the agitator and the separation device inlet was studied at a speed of 60 rpm, for both downward and upward pumping with a 13 d-old cell suspension. The cell-free liquid height at the top of the device was then measured. Surprisingly, the lower was the column-impeller distance (1 cm), the lower a stable front was formed (12 cm), with high separation efficiency (data not shown). This position corresponded to a device immersion of 7 cm. The effect of impeller rotational direction and speed were evaluated at that immersion position (Table 1). The perturbations of the cell front increased with the agitator rotational speed, but downward pumping induced lower front perturbations. The use of a cross at the column inlets increased the front's stability.

TABLE 1
Influence of the operating conditions on the front
stability for a 13 day-old cell suspension, with a 1
cm distance between column type D and top agitator

| | Agitator | | |
|---|---|---|---|
| Cross | speed (rpm) | Upwards pumping | Downwards pumping |
| no | 25, 30, 35 | partial cell | stable front |
| | | entrainment | |
| yes | 30 | stable front | stable front |
| no | 40, 60 | high cell loss | high cell loss |
| yes | 40, 60 | medium cell loss | stable front |

* To avoid any influence of the pumping and time, the flow rate was stopped once the column was full of liquid. The front was considered to be stable when there was a minimum liquid height of 5 cm.

### Position of the Gas Sparger

Injection of gas at the bottom of the bioreactor caused bubbles to enter the devices, which destabilized the front and seemed to change the columns into perfectly mixed zones. Positioning the gas sparger at 1 cm above columns inlet has reduced the entering of gas bubbles into the columns to almost zero. The effect of having the sparger at the top of the bioreactor on the oxygen transfer rate was then evaluated (FIG. 4). Agitator rotational direction and speed showed little influence on the kla as compared to the gas sparger position. The expected maximum OUR for a 10 gDW.l⁻¹ cell suspension of E. californica is of 2.43 mM O₂.g⁻¹ DW (Lamboursain et al., 2002) which implies a required kla of 21.6 h⁻¹ with air feed and 4.6 h⁻¹ feeding pure oxygen. Despite of a clear decrease in the bioreactor's potential to transfer oxygen to the cells when bubbling gas at 6 cm from the top liquid level, it was estimated that the use of an oxygen-enriched gas could result in a sufficient oxygen transfer (FIG. 4).

### Preliminary Experiments in the Perfusion Bioreactor

Experiments based on the use of a bioreactor culture without extraction columns were performed to determine the evolution of the sedimentation velocity with time as well as to fine-tune the perfusion process. The operating conditions were those determined as optimal by the hydrodynamic studies with 4 D-type separation devices equipped with an inlet cross and immersed at 1 cm from the agitator, and the agitation at 40 rpm with downwards pumping. The culture was elicited on day 4 as described for the perfusion bioreactor. The sedimentation velocity of each sample withdrawn during the culture was measured and the perfusion flow rate was readjusted manually in order to keep a stable bed height in the column (Table 2). Very high perfusion rates were reached without measurable cell lost at SCV of up to 50%. A perfusion rate of 22 d⁻¹ was maintained from day 4 to 6, which had to be decreased at 11.7 d⁻¹ on day 7 and 9.8 d⁻¹ on day 8 because of a change in the cell suspension quality or content, as discussed previously. These values are on order magnitude higher than that found in literature (Kim et al., 1991; Su and Arias, 2003; Su et al., 1996). However, in order to reduce risks of cell entrainment, a safety margin was established with the flow rates set arbitrarily 5 ml.min⁻¹ lower than the flow rates determined by the previous experiments. At days 4, 5 and 6, the flow rate was then set to 45 ml.min⁻¹ (20.4 d⁻¹), and for days 7 and 8, it was set to 16 ml.min⁻¹ (7 d⁻¹). The sedimentation velocity for day 9 was not measured but estimated to be about 3 mm.min⁻¹, which corresponds to a flow rate of 16 ml.min⁻¹. To be cautious, the flow rate was set to 11 ml.min⁻¹ (5 d⁻¹). The extraction columns were then designed from the estimated pumping rates and the extractive resin fluidization velocity. The three columns to be used successively were determined as column I (185 mm×26 mm, I.D.) on days 4, 5 and 6, column II (240 mm×17 mm I.D.) on days 7 and 8, and column III (330 mm×13 mm I.D.) on day 9. The perfusion bioreactor was then tested with the use of the extraction columns.

TABLE 2
Sedimentation velocity and pumping flow rate vs time and SCV
during the preliminary experiment in the perfusion bioreactor

| | Culture | Sedimentation | | |
|---|---|---|---|---|
| SCV | time | velocity | Pumping flow rate | Perfusion rate |
| (%) | (d) | (mm · min⁻¹) | (ml · min⁻¹) | (d⁻¹) |
| 33 | 4 | 9.2 | 48.6 | 21.9 |
| 34 | 5 | 9.7 | 51.2 | 23.0 |
| 32 | 6 | 9.6 | 50.5 | 22.7 |
| 47 | 7 | 4.9 | 26.0 | 11.7 |
| 46 | 8 | 4.1 | 21.7 | 9.8 |

* The culture was performed with optimal operating conditions (4 D-type columns, inlet cross, agitator downwards pumping, 1 cm between top of agitator and wet end of the columns).

### Medium Perfusion and Continuous Extraction of Secondary Metabolites do not Affect Cell Growth and Nutrition in a Bioreactor

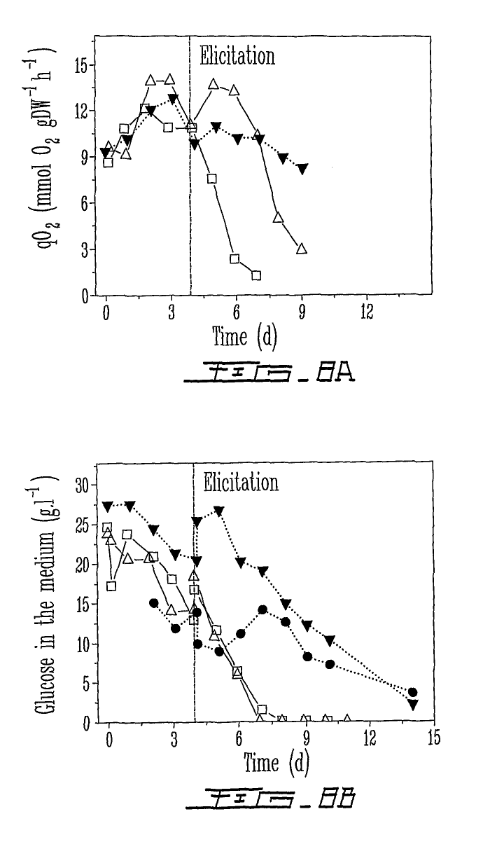

The perfusion cultures with the extraction columns resulted in a similar growth rates (0.23 d⁻¹) than cultures with free resins and that without XAD-7 resins (Table 3). Despite higher inocula for the perfusion cultures, the growth index evolved similarly for all cultures before elicitation (FIG. 5A). Then, addition of fresh medium and of the chitin extract solution at elicitation (day 4) has perturbed the cultures at different levels. For the non-perfused bioreactor, medium addition at elicitation has to cover the volume decrease from sampling, but for the perfusion bioreactor, a supplementary volume was required to fill the recirculation loop. Liquid addition caused a 16% cell dilution for the cultures with free resins and without resins. In the perfusion bioreactor, cells experienced an actual dilution of 38%. However, the apparent dilution level measured in the bioreactor was artificially of 20% since the sedimentation devices were efficient and retained the cells within the bioreactor, leaving the extra recirculation loop of 860 ml free of cells. The extra medium addition seemed to have delayed biomass growth by 2 days, among other factors discussed below. However, similar growth rates of 0.23±0.04 d⁻¹ were also observed after elicitation for all the culture strategies and at values that were similar than those before elicitation. A similar maximum dry biomass of 1 5.9±2.8 gDW.l⁻¹ was reached from day 7 to day 9. The evolution of the WW/FW ratio was also similar in all cultures, showing an increase from day 9 (FIG. 5B).

TABLE 3
E. californica maximum specific growth rate calculated before and
after elicitation for the different culture strategies in bioreactor (with
and without resin), as in the bioreactor with perfusion system

μₘₐₓ* (d⁻¹)

| Bioreactor culture | Pre-elicitation | Post-elicitation |
|---|---|---|
| No extractive phase | 0.22 ± 0.02 | 0.20 ± 0.04 |
| XAD-7 resins (n = 2) | 0.25 ± 0.02 | 0.27 ± 0.02 |
| Medium perfusion (n = 2) | 0.20 ± 0.02 | 0.23 ± 0.05 |

*μₘₐₓ was calculated in the exponential phase using a minimum of 3 data points

### Production of Secondary Metabolites*

Cultures grown with free resins showed maximum global secondary metabolites with concentration of 30.94 μmole.gDW⁻¹, which were distributed as 28.4±8.8 μmole.gDW⁻¹ in the resins and 2.54 μmole.gDW⁻¹ in the cells. The culture without any extractive resins has reached 2.25 μmole.gDW⁻¹ intracellularly. Alkaloids content of all culture media were under the detection limit of the analytical. It is well documented that the addition of extractive resins in a cell suspension results in productivities that are 10 (Byun et al., 1990; Collinge and Brodelius, 1989; Klvana et al., 2004) to 30 (Byun and Petersen, 1994; Villegas et al., 2000) times that measured without resin.

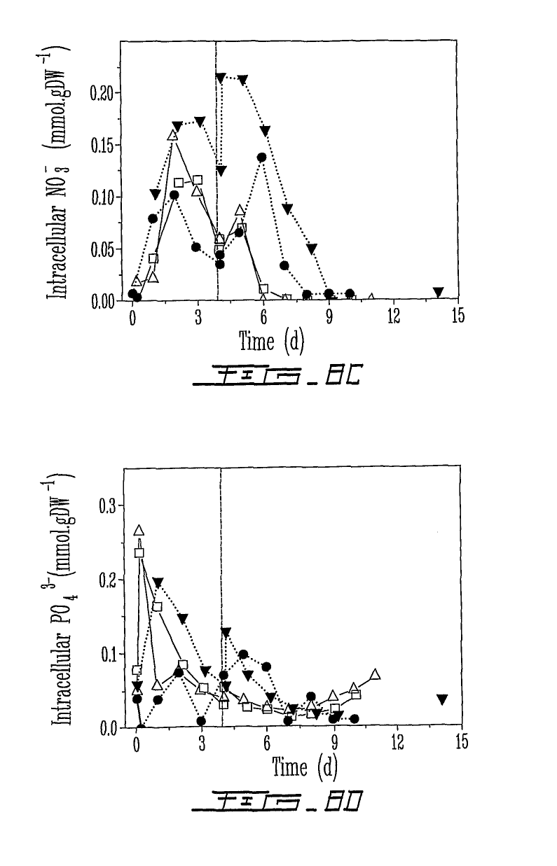

For the perfusion bioreactor, a maximum total alkaloid content of 2.06 μmole.gDW⁻¹ was measured with 1.54 μmole.gDW⁻¹ extracted in the resin (FIG. 6) and a cell content of 0.52 μmole.gDW⁻¹ (FIG. 7). Interestingly, the perfusion bioreactor cultures with no direct contact between the resins and the cells showed to favor the chelelutine pathway, and cultures without resins and with free resins favored the macarpine pathway. For the cultures with free resins (FIG. 7), macarpine was dominant with 60% of the total alkaloids adsorbed and the pathway leading to macarpine (sanguinarine, chelerythrine and macarpine) accounted for 80 to 85% of the total adsorbed alkaloids. For resins in the extractive columns there was between 60 to 70% of the alkaloids as chelerubine and chelelutine. Overall, macarpine was dominant with 55 to 65% of the total alkaloids for the cultures with free resins, and chelelutine was dominant with 55 to 60% for the perfusion cultures. This change in the fluxes of the secondary metabolism has been recently reported by Klvana et al. (2004) for shake flask culture with extractive column. However, the production levels in alkaloids were similar for the flasks with free resins and those equipped with extractive columns. In flask equipped with an extractive column 63% of the total alkaloids was of the macarpine pathway as compared to 64% of the chelelutine pathway (58% chelelutine and 29% macarpine) when using an extractive column. In the present work, it was surprising to observed the same change in the metabolism even if the productivity level in the perfusion bioreactor was significantly lower that that with free resins. This result may thus confirm the hypothesis that a direct cell/resins contact favors the macarpine pathway and that an indirect adsorption of secondary metabolites favors the chelelutine pathway (Klvana et al., 2004).

It was surprising that the productivities in the perfusion bioreactor were low and at the levels of the culture without resins. Some of the operating conditions in the perfusion bioreactor have thus significantly reduced the global performance potential of the culture. Klvana et al. (2004) reported total contents in alkaloids of 4.66, 4.94 and 6.8 μmol.g DW⁻¹ 6 days after elicitation in flask culture with the same E. californica cell line, respectively, for cultures without resin, with resin and with a recirculation column containing resin. Intracellular contents in secondary metabolites in Klvana et al. (2004) were similar to this work with 1.18 (without resin), 0.12 (with resin) and 1.53 μmol.g DW⁻¹ (column containing resin). A similar value of 0.8 μmole.gDW⁻¹ (per day) was observed by Byun et al. (1992) for Eschscholtzia californica with yeast extract as elicitor.

The addition of anti-foam has also first be pointed out as a possible cause for the lower productivities by possibly affecting resins adsorption capacity. A second perfusion culture was established without any anti-foam addition and resulted in productivities slightly lower than with the use of anti-foam (not shown data). In the perfusion bioreactor, lower amounts of resins were continuously in contact with the recirculation medium. The three columns used successively contained 29.8, 23.6 and 17.4 g (70.8 g total) of resins as compared to 150 g of resin in the bioreactor with free resins. The resin-to-medium ratios were of 8.5, 6.7 and 5 g.l⁵¹ for the perfusion bioreactor as compared to 50 g.l⁻¹ for the bioreactor with free resins and 40 g.l⁻¹ for the flask culture (Klvana et al., 2004). It has been proposed by Klvana et al. (2004) that this resin-to-cell suspension ratio may be important.

In this work, results showed that the chelelutine pathway was favored in the perfusion bioreactor as in the work of Klvana et al. (2004) who used higher resin-to-cell suspension ratio. Therefore, the resin-to-cell suspension ratio may be an important parameter but since the resins were far from saturation in secondary metabolites in this work, it is thought that the level in cell contact with extractive resins plays on the metabolic pathways flux distribution but that another parameter is involved in the control of the production level in secondary metabolites as discussed below. The use of a higher amount of resins may allow a longer contact time for the metabolites and the resins.

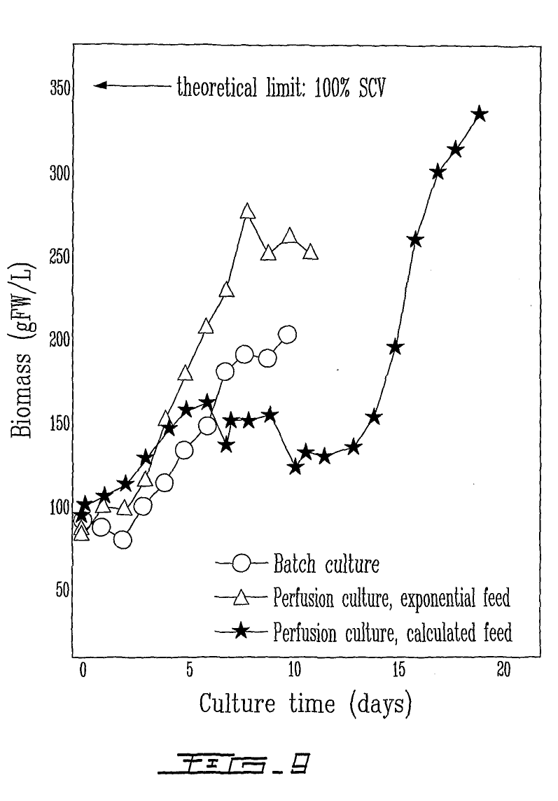

A higher cell dilution with fresh medium at elicitation may have also affected the productivities in the perfusion culture because of the extra volume of the recirculation loop. Indeed, it has recently been shown that the identity of the limiting nutrient plays a crucial role in cell growth potential and secondary metabolites production (Lamboursain and Jolicoeur, 2005). In these references, it was shown with the same cell line that the maximum potential for the cells to produce secondary metabolites was from day 1 to day 3 and from day 6 to day 10, respectively, for Pi-limited and nitrogen (N)-limited cells. Then, moving between Pi and N limitations, the lowest cell productivity potential is around day 4, the time at which elicitation was systematically performed here. Surprisingly, the difference between the minimum and the maximum production levels was of 10×, same as observed between the culture with suspended resin and the perfusion culture. Therefore, the physiological state of the initial inocula was certainly similar but this was no more true from elicitation since the cell suspensions were submitted to different dilution levels and nutrients replenishments. This has resulted in discrepancies between the nutritional state of the cell suspensions (FIG. 8). It is clear that the cells in the perfusion bioreactor were the less stressed by nutrient limitations. Medium glucose was not totally uptaked during the 14 d culture and intracellular contents in nitrate and inorganic phosphate were high until day 9. The results are in agreement with the hypothesis proposed by Lamboursain and Jolicoeur (2005) but this has to be further investigated.

### Example 3

### Production and In-Situ Extraction of a Recombinant Protein Using a Plant Cell System

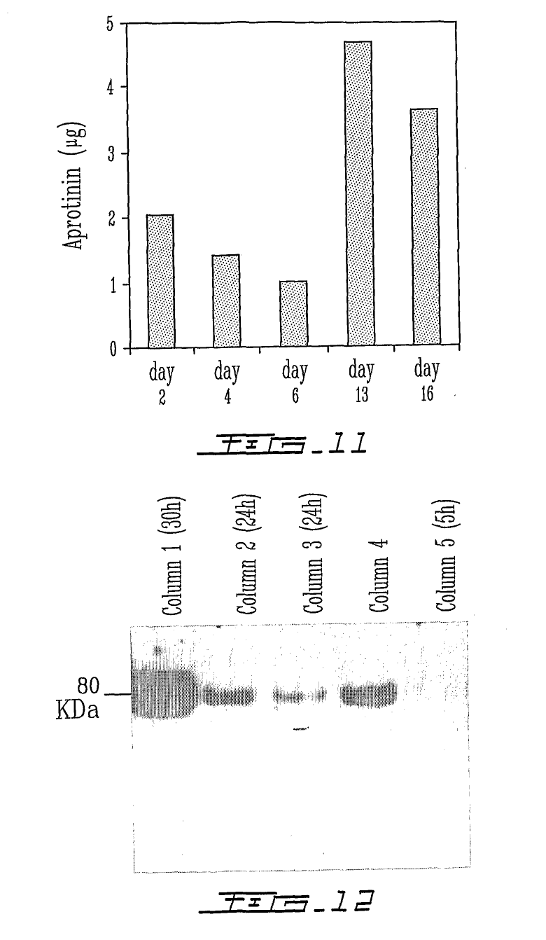

The bioreactor was used efficiently to demonstrate its ability for protein production using plant cell lines. Alfalfa cells genetically modified to produce recombinant aprotinin were cultured for 20 days. Medium was aseptically recirculated through a single sedimentation column at a perfusion rate of 2 d⁻¹ from day 5. Despite the use of a cell line for which the genetic modifications as well as the cell line selection were not optimized cell suspension culture neither for protein secretion; accumulation of aprotinin was observed in the extracellular medium before medium recirculation. Recirculated medium was fed through a fluidized bed of affinity resins for protein extraction before to be returned back to the bioreactor vessel. The extraction phase was composed of a Sepharose™ matrix coupled to trypsin, a natural ligand for aprotinin. Thus from day 5 and medium perfusion, aprotinin accumulated in 5 successive extraction columns for a total amount of 12.8 μg (FIG. 11). Column 1 was installed for two days from day 0 to day 2 under medium perfusion (day 5); column 2 was installed from day 2 to day 4; column 3 was installed from day 4 to day 6; column 4 was installed from day 6 to day 13; column 5 was installed from day 13 to day 16. Aprotinin concentration reached 2.2 μg ml⁻¹ in the extracellular medium on day 4, the day before the beginning of the continuous in-situ extraction. Then, aprotinin concentration in the extracellular medium dropped at undetectable level for the time when using affinity columns. Thus, all the secreted recombinant aprotinin were captured on the affinity column. These results showed that the perfusion bioreactor is especially designed for the capture or recombinant proteins which are secreted even at a very low level into the culture medium.

### Example 4

### In-Situ Extraction of an Endogenous Secreted Protein

Tobacco cells were cultured in the perfusion bioreactor. The perfusion system was started at day 5. A perfusion rate of 7 d⁻¹ was applied and allowed recirculation of the culture medium through affinity columns. Theses extractions columns were operated in a fluidized-bed mode with nickel-charged particles traditionally used for the capture of chimerical poly-histidine tag proteins. Although no recombinant proteins were secreted by the tobacco cell line used, extraction of an endogenous protein showing a high affinity for the Ni-charged resins was observed. This protein was only detected after elution of the resins (FIG. 12). It was then detected by an anti-6His antibody in a subsequent Western blot analyses. After peptide mapping of the gel-purified protein, this protein appeared to belong to the family of the β-xylanases, enzymes which are implicated in the maturation of the cell wall.

The protein found on the extraction columns was not detected in the extracellular medium samples from the bioreactor, even when concentrated 50×. This result also suggests the high efficiency of the system to recover the secreted protein.

### Example 5

### Use of the Perfusion Bioreactor with Adherent Animal Cells

Culture of adherent animal cells on porous microcarriers allows achieving a high cell density and high rate medium perfusion. Nutrients renewal and the continuous removal of desired products with the affinity columns as well as removing the undesired metabolic by-products can then be achieved in the perfusion bioreactor. Activated microcarriers (200 mL Cytoline 1, GE Healthcare) were used. Agitation by the double helical ribbon impeller allows for a good mixing of the microcarriers suspension and facilitates the transfer of the nutrients to the cells (including oxygen supply). A series of tests were performed at agitation speeds of 30, 60, 90 rpm. A minimum agitation of 60 rpm is required to maintain the microcarriers in suspension. A medium perfusion rate of 22 mL min⁻¹ per sedimentation column was applied without entraining any microcarrier particles. Thus the total perfusion flow rate for the culture using the four sedimentation columns can be as high as 88 mL min⁻¹, which corresponds to a dilution rate of 1.6 h⁻¹ or a residence time of 0.63 h, and is largely sufficient to support the very high cell density required in recombinant protein production.

A perfusion bioreactor allowing high perfusion rates was designed and challenged with E. californica plant cell suspension culture. Perfusion started at the time of elicitation (day 4) at a rate of 20.4 d⁻¹ and has to be regularly lowered to 5 d⁻¹ at the end of the culture because of a high level in cell debris. These rates are 2.5 to 10 times those reported in literature for the separation of plant cells and medium in a bioreactor. However, the cultures performed in the perfusion bioreactor showed productivities in secondary metabolites that were 10 times lower than that obtained in cultures with free resins. Based on recent results (Lamboursain and Jolicoeur, 2005) it was hypothesized that the lower productions in the perfusion bioreactor were due to operating conditions which induce major differences in the cells nutritional state following elicitation. Further studies will then be conducted to identify an adequate strategy for cell elicitation which can maintain optimal cell nutritional conditions. Studies with other plant species have to be conducted in order to address the capacity of the perfusion bioreactor industrially. Preliminary assays performed with Nicotiana tabacum seemed to confirm the applicability of the bioreactor to that cell suspension as well (data not shown). In the bioreactor design presented in this work, the extraction column can be rapidly replaced and treated during a culture. This feature allows to extend the time of production and to remove from the used culture medium metabolites showing short half-lives. It also simplifies significantly the downstream processes by reducing the required purification steps. This new bioreactor is of high potential for large scale production of biomolecules such as secondary metabolites and recombinant proteins.

Production of metabolites of interest through a perfusion bioreactor offers many advantages compared to batch solutions and the perfusion systems developed earlier. The separation by sedimentation between cell and medium imposes low shear stress and is not subject to clogging because it does not use any membrane. The bioreactor permits high circulation rates (20.4 d⁻¹ at the beginning to 5 d⁻¹ at the end of the culture) and thus high extraction levels. These rates are high in comparison to values found in the literature for the separation of plant cells and medium in a bioreactor. However, alkaloid production was low due to the relatively poor performance of the extraction columns. Nevertheless, this system is a great alternative to immobilized plant cell bioreactors that are currently in use, and shows great potential for the large-scale culture of plant cells.

Although the present invention has been described hereinabove by way of preferred embodiments thereof, it can be modified without departing from the spirit, scope and nature of the subject invention, as defined in the appended claims.

### LIST OF REFERENCES

1. Batt B C, Davis R H, Kompala D S. 1990. Inclined sedimentation for selective retention of viable hybridomas in a continuous suspension bioreactor. Biotechnol Prog 6:458-464.
2. Byun S Y, Pedersen H, Chin C K. 1990. Two-phase culture for the enhanced production of benzophenanthridine alkaloids in cell suspensions of Eschscholtzia californica. Phytochemistry 29: 3135-3139.
3. Byun S Y, Kim C, Pedersen H. 1992. Elicitation of alkaloid production at different growth stages in cell suspensions of Eschscholtzia californica. Plant Tissue Culture Letters 9:164-168.
4. Byun S Y, Pedersen H. 1994. Two-phase airlift fermentor operation with elicitation for the enhanced production of benzophenanthridine alkaloids in cell suspensions of Eschscholtzia californica. Biotechnol Bioeng 44: 14-20.
5. Collinge M A, Brodelius P E. 1989. Dynamics of benzophenanthridine alkaloid production in suspension cultures of Eschscholtzia californica after treatment with a yeast elicitor. Phytochemistry 28: 1101-1104.
6. Chattopadhyay S, Bisaria V S, Srivastava A K. 2003. Enhanced production of podophyllotoxin by Podophyllum hexandrum using in situ cell retention bioreactor. Biotechnol Prog 19:1026-1028.
7. Daniell H, Streatfield S J, Wycoff K. 2001. Medical molecular farming: production of antibodies, biopharmaceuticals and edible vaccines in plants. Trends Plant Sci 6:219-226.
8. Doran P M. 1999. Design of mixing systems for plant cell suspensions in stirred reactors. Biotechnol Prog 15:319-335.
9. Doran P M. 2000. Foreign protein production in plant tissue cultures. Curr Opin Biotechnol 11:199-204.
10. Gamborg O L, Miller R A, Ojima K. 1968. Nutrient requirements of suspension cultures of soybean root cells. Exp Cell Res 50:151-8.
11. Gmati D, Chen J, Jolicoeur M. 2004. Development of a Small-Scale Bioreactor: Application to In Vivo NMR Measurement. Biotechnol and Bioeng In press.
12. Gorenflo V M, Angepat S, Bowen B D, Piret J M. 2003. Optimization of an acoustic cell filter with a novel air-backflush system. Biotechnol Prog 19:30-36.
13. Hogue R S, Lee J M, Ant G. 1990. Production of a foreign protein product with genetically modified plant cells. Enzyme Microb Technol 12:533-538.
14. Horn M E, Woodard S L, Howard J A. 2004. Plant molecular farming: systems and products. Plant Cell Rep 22:711-720.
15. Hülscher M, Scheibler U, Onken U. 1992. Selective recycle of viable animal cells by coupling of airlift reactor and cell settler. Biotechnol Bioeng 39:442-446.
16. Johnson M, Lanthier S, Massie B, Lefebvre G, Kamen A A. 1996. Use of the Centritech Lab centrifuge for perfusion culture of hybridoma cells in protein-free medium. Biotechnol Prog 12:855-864.
17. Jolicoeur M, Chavarie C, Carreau P J, Archambault J. 1992. Development of a helical-ribbon impeller bioreactor for high-density plant cell suspension culture. Biotechnol Bioeng 39:511-521.
18. Kawahara H, Mitsuda S, Kumazawa E, Takeshita Y. 1994. High-Density Culture of Fm-3a Cells Using a Bioreactor With an External Tangential-Flow Filtration Device. Cytotechnol 14:61-66.
19. Kim D I, Cho G H, Pedersen H, Chin C K. 1991. A hybrid bioreactor for high density cultivation of plant cell suspensions. Appl Microbiol and Biotechnol 34:726-729.
20. Klvana M, Legros R, Jolicoeur M. 2004. In situ extraction strategy affects benzophenanthridine alkaloid production fluxes in suspension cultures of Eschscholtzia californica. Biotechnol Bioeng, 89:280-289
21. Klvana M, Chen J, Lépine F, Legros R, Jolicoeur M. Screening of Eschscholtzia californica secondary metabolites by high-performance liquid chromatography. Phytochem Anal In press.
22. Lamboursain L, Jolicoeur M. 2005. Critical influence of Eschscholtzia californica cells nutritional state on secondary metabolite production. Biotechnol Bioeng 91:827-837.
23. Lamboursain L, St-Onge F, Jolicoeur M. 2002. A lab-built respirometer for plant and animal cell culture. Biotechnol Prog 18:1377-1386.
24. Lee S Y, Kim Y H, Roh Y S, Myoung H J, Lee K Y, Kim D I. 2004. Bioreactor operation for transgenic Nicotiana tabacum cell cultures and continuous production of recombinant human granulocyte-macrophage colony-stimulating factor by perfusion culture. Enzyme Microb Technol 35:663-671.
25. Lee-Parsons C W T, Shuler M L. 2002. The effect of ajmalicine spiking and resin addition timing on the production of indole alkaloids from Catharanthus roseus cell cultures. Biotechnol Bioeng 79:408-415.
26. Ramachandra R S, Ravishankar G A. 2002. Plant cell cultures: Chemical factories of secondary metabolites. Biotechnol Advances 20:101-153.
27. Richardson J F, Zaki W N. 1954. Sedimentation and Fluidisation. Trans. Inst. Chem. Eng., 32:35-53.
28. Ryu D D Y, Lee S O, Romani R J. 1990. Determination of growth rate for plant cell cultures: comparative studies. Biotechnol Bioeng 35:305-311.
29. Searles J A, Todd P, Kompala D S. 1994. Viable cell recycle with an inclined settler in the perfusion culture of suspended recombinant chinese hamster ovary cells. Biotechnol Prog 10:198-206.
30. Seki M, Ohzora C, Takeda M, Furusaki S. 1997. Taxol (paclitaxel) production using free and immobilized cells of Taxus cuspidata. Biotechnol. Bioeng. 53:214-219.
31. Su W W, Arias R. 2003. Continuous plant cell perfusion culture: Bioreactor characterization and secreted enzyme production. J Biosci and Bioeng 95:13-20.
32. Su W W, He B J, Liang H, Sun S. 1996. Perfusion air-lift bioreactor for high density plant cell cultivation and secreted protein production. J Biotechnol 50:225-233.
33. Tanaka H. 2000. Technological problems in cultivation of plant cells at high density. Biotechnol Bioeng 67:775-790.
34. Tikhomiroff C, Allais S, Klvana M, Hisiger S, Jolicoeur M. 2002. Continuous selective extraction of secondary metabolites from Catharanthus roseus hairy roots with silicon oil in a two-liquid-phase bioreactor. Biotechnol Prog 18:1003-1009.
35. Villegas M, Sommarin M, Brodelius P E. 2000. Effects of sodium orthovanadate on benzophenanthridine alkaloid formation and distribution in cell suspension cultures of Eschscholtzia californica. Plant Phys Biochem 38: 233-241.
36. Voisard D, Meuwly F, Ruffieux P A, Baer G, Kadouri A. 2003. Potential of cell retention techniques for large-scale high-density perfusion culture of suspended mammalian cells. Biotechnol Bioeng 82:751-765.
37. Williams R D, Chauret N, Bedard C, Archambault J. 1992. Effect of Polymeric Adsorbents on the Production of Sanguinarine by Papaver-Somniferum Cell-Cultures. Biotechnol Bioeng 40:971-977.

1. A perfusion bioreactor apparatus comprising:
a vessel for receiving a cellular biomass suspension comprising cells and a cellular medium therein, and;
an extractor assembly mounted to the vessel for extracting the cellular medium from said vessel;
wherein said extractor is adapted to extract the cellular medium substantially avoiding extraction of the agitated cellular biomass.

2. The perfusion bioreactor apparatus of claims 1, wherein the cellular medium comprises cellular product, the extractor assembly being adapted to extract the cellular medium along with the cellular product.

3. The perfusion bioreactor apparatus of claim 1, wherein said extractor comprises at least one sedimentation column defining a channel and being mounted within the vessel.

4. The perfusion bioreactor apparatus according to claim 3, wherein the sedimentation column is mounted in the vessel along the longitudinal length thereof.

5. The perfusion bioreactor apparatus according to claim 4, wherein the channel is so configured as to provide for a separation of the cellular medium and the cells when the cellular biomass enters the channel.

6. The perfusion bioreactor apparatus of claim 3, wherein the sedimentation column comprises an inlet opening configured to be located inside the vessel and an outlet opening configured to be located outside the vessel.

7. The perfusion bioreactor apparatus of claim 3, wherein the sedimentation column has a generally cylindrical configuration.

8. The perfusion bioreactor apparatus of claim 3, wherein the sedimentation column has a generally funnel shape.

9. The perfusion bioreactor apparatus of claim 3, wherein the sedimentation column comprises a vortex reducer at an inlet opening thereof.

10. The perfusion bioreactor apparatus of claim 3, further comprising an agitator assembly, the agitator assembly comprising an impeller for mounting within the vessel, the sedimentation column having an inlet opening thereof configured to be located near the impeller.

11. The perfusion bioreactor apparatus of claim 3, wherein the sedimentation column is in fluid communication with a cell sedimentation module.

12. The perfusion bioreactor apparatus of claim 3, wherein the sedimentation column is mounted to a pump for pumping the cellular medium therethrough from an inlet opening within the vessel to an outlet opening outside the vessel.

13. The perfusion bioreactor apparatus of claim 1, wherein the extraction assembly provides for a continuous extraction of the cellular medium.

14. The perfusion bioreactor apparatus of claim 1, wherein the extractor assembly comprises at least one extraction column.

15. The perfusion bioreactor apparatus of claim 3, wherein the extractor assembly comprises at least one extraction column, the extraction column being in fluid communication with the sedimentation column.

16. The perfusion bioreactor apparatus of claim 15, further comprising a cell sedimentation module in fluid communication with both the extraction column and the sedimentation column.

17. The perfusion bioreactor apparatus of claim 16, wherein the extraction column is in fluid communication with the vessel, thereby defining a fluid circuit from the vessel to the sedimentation column, to the cell sedimentation module, to the extraction column and to the vessel.

18. The perfusion bioreactor apparatus of claim 14, wherein the extraction column is in fluid communication with the vessel.

19. The perfusion bioreactor apparatus of claim 14, comprising a first and second extraction column, one of said first or second column having an internal diameter lower than an internal diameter of the other of said first or second column.

20. The perfusion bioreactor apparatus of claim 14, wherein the extraction column further comprises a separating means or a capturing means.

21. The perfusion bioreactor apparatus of claim 20, wherein the separating means or capturing means is fluidized.

22. The perfusion bioreactor apparatus of claim 20, wherein the separating means or the capturing means is selected from the group consisting of an affinity matrix, an absorbent resin, a size-exclusion matrix and an ion-exchange matrix.

23. The perfusion bioreactor apparatus of claim 1, further comprising an agitator assembly for mounting within the vessel for so agitating the cellular biomass suspension within the vessel as to provide a stable sedimentation front.

24. The perfusion bioreactor apparatus of claim 23, wherein the agitator assembly comprises an impeller.

25. The perfusion bioreactor apparatus of claim 24, wherein the impeller is helical.

26. The perfusion bioreactor apparatus of claim 23, wherein the agitator assembly is adapted to provide an upward movement to the cellular medium within the vessel.

27. The perfusion bioreactor apparatus of claim 23, wherein said agitator assembly is adapted to provide a downward movement to the cellular medium within the vessel.

28. The perfusion assembly of claim 1, further comprising an air and/or gas entry means for providing air and/or gas into the vessel.

29. The perfusion assembly of claim 28, wherein the extractor assembly comprises a sedimentation column having an inlet opening thereof mounted within the vessel, the air and/or gas entry means being located above the inlet opening and below a liquid surface within the vessel

30. The perfusion assembly of claim 1, further comprising an air and/or gas exit means for providing air and/or gas to exit the vessel.

31. The perfusion assembly of claim 1, further comprising a liquid entry means for providing liquid into the vessel.

32. The perfusion assembly of claim 1, further comprising a liquid exit means for providing liquid to exit the vessel.

33. The perfusion assembly of claim 1, wherein the extractor assembly is adapted to extract the cellular medium from the cellular biomass suspension at an extraction velocity rate equal to or lower than a sedimentation velocity of the cellular biomass suspension.

34. The perfusion bioreactor apparatus of claim 1, wherein said apparatus is adapted for a continuous feed of cell culture medium.

35. The perfusion bioreactor apparatus of claim 1, for use in growing cells and/or for producing a cellular product from cells.

36. A process for the continuous extraction of a cellular product contained in a cellular medium said process comprising:
providing a cellular biomass suspension comprising cells and cellular medium in agitation;
providing for the production of a cellular product from the cellular biomass suspension;
providing a stable sedimentation front;
extracting the cellular medium with the cellular product from the cellular biomass suspension at an extraction velocity rate equal to or lower than a sedimentation velocity of the cellular biomass suspension thereby substantially avoiding extraction of the cellular biomass.

37. The process of claim 1, further comprising providing for the separation of the cellular medium and the cells before said extracting.

38. The process of claim 36, wherein the cellular medium is continuously removed from the vessel.

39. The process of claim 38, comprising continuously removing the cellular product from the cellular medium.

40. The process of claim 36, wherein the sedimentation velocity of the cellular biomass suspension is determined in a linear region of a slope made by measuring a cell bed height decrease rate as a function of time.

41. The process of claim 40, wherein the sedimented cell volume is used for determining the sedimentation velocity of the cellular biomass suspension.

42. The process of claim 36, wherein said sedimented cell volume is 80% or less.

43. The process of claim 36, wherein said sedimented cell volume is 75% or less.

44. The process of claim 36, wherein said sedimented cell volume is 70% or less.

45. The process of claim 36, wherein the sedimented cell volume is at least 80%.

46. The process of claim 36, wherein the sedimented cell volume is at least 90%.

47. The process of claim 36, wherein the cellular medium is reinserted into the vessel following removal of product.

48. The process of claim 36, wherein agitation is performed in a manner allowing upward flowing of cellular medium.

49. The process of claim 36, wherein agitation is performed in a manner allowing downward flowing of cellular medium.

50. The process of claim 36, wherein said process comprises reducing an agitation vortex.

51. The process of claim 36, wherein the product is removed from the cellular medium by contacting the cellular product with a separating means or a capturing means.

52. The process of claim 51, wherein the separating means or the capturing means is selected from the group consisting of an affinity matrix, an absorbent resin, a size-exclusion matrix and an ion-exchange matrix.

53. The process of claim 36, wherein said cells are selected from the group consisting of mammalian cells, plant cells, insect cells and bacterial cells.

54. The process of claim 53, wherein the cells are able to be grown in suspension.

55. The process of claim 54, wherein the cells are adherent.

56. The process of claim 55, wherein the adherent cells are grown on micro-carriers.

57. The process of claim 53, wherein the plant cells are selected from the group consisting of a tobacco cell, a flower cell, an alfalfa cell, a maize cell, a canola cell, a safflower cell, a rice cell, a barley cell, a carrot cell.

58. The process of claim 53, wherein the plant cells are Eschscholtzia californica cells or Nicotiana tabacum cells.

59. The process of claim 36, wherein the cells express a native protein or a recombinant protein.

60. The process of claim 59, wherein the native protein or recombinant protein is secreted in the cell culture medium.

61. The process of claim 36, wherein the cells produce metabolites.

62. The process of claim 61, wherein the metabolites include an alkaloid.

63. The process of claim 62, wherein the alkaloid is induced by addition of an elicitor in the cell culture medium.

64. The process of claim 62, wherein the alkaloid is a benzophenanthridine.

65. The process of claim 64, wherein the benzophenanthridine is selected from the group consisting of sanguinarine, chelerythrine, chelerubine, chelilutine and macarpine.

66. The process of claim 36, wherein the process is performed in the dark.

67. The process of claim 36, wherein the process is performed under aseptic conditions.

68. The process of claim 36, wherein an oxygen-enriched gas is provided to the cellular biomass.

69. The cellular product produced by the process of claim 36.

70. The cellular product extracted by the process of claim 36.
